(19) United States
(12) Patent Application Publication
(10) Pub. No.: US 2015/0240233 A1
(43) Pub. Date: Aug. 27, 2015

(54) METHODS FOR IDENTIFYING PLANT PATHOGEN RESISTANCE GENES

(71) Applicant: Two Blades Foundation, Evanston, IL (US)

(72) Inventors: Jonathan Jones, Norwich (GB); Burkhard Steuernagel, Norwich (GB); Brande Bruce Hertel Wulff, Norwich (GB)

(21) Appl. No.: 14/627,116

(22) Filed: Feb. 20, 2015

Related U.S. Application Data
(60) Provisional application No. 61/942,771, filed on Feb. 21, 2014.

(51) Int. Cl. C12N 15/10

(52) U.S. Cl. CPC C12N 15/1079

(57) ABSTRACT

Methods are provided for identifying a plant disease resistance (R) gene for a plant disease of interest. The methods involve using bait sequences to select a subgroup of nucleic acids from a group of nucleic acids that are derived from a mutagenized plant that is susceptible to the plant disease of interest but that was produced by mutagenizing a plant that is resistant to the disease of interest. The bait sequences are designed to hybridize to one or more genes from at least one plant R gene family. The methods further involve sequencing the subgroup of nucleic acids to obtain a collection of nucleic acid sequences, comparing such nucleic acid sequences with corresponding sequences of one or more genes that are derived from a resistant plant, and identifying at least one nucleic acid sequence derived from the mutagenized plant that is not identical in sequence to a corresponding sequence from the resistant plant. Further provided are related methods for identifying a gene associated with a phenotypic change for a trait of interest in plants and other organisms.

### CROSS-REFERENCE TO RELATED APPLICATION

This application claims the benefit of U.S. Provisional Application No. 61/942,771, filed Feb. 21, 2014, which is hereby incorporated herein in its entirety by reference.

### FIELD OF THE INVENTION

The present invention relates to the field of molecular biology and genetics, particularly to methods for identifying genes for traits of interest.

### BACKGROUND OF THE INVENTION

Plant disease causes significant yield losses in agriculture. Wheat and potato are two of the most important crops worldwide, including India and the United Kingdom. Among the most damaging diseases of wheat are the rusts. Stripe rust occurs wherever the crop is grown causing average yearly yield losses of up to 10% in some regions. Stem rust was until the green revolution associated with regular crop failures and famine. The resistance introduced then has now been broken by new strains of the fungus, which started appearing in Africa 14 years ago. The potato late blight disease, the cause of the Great Irish Potato famine in the 1840s, is still a serious impediment to potato cultivation today. Pesticides can control these diseases but they are expensive, at odds with sustainable intensification of agriculture, and in developing countries and for subsistence farmers, they are simply unaffordable.

Wild relatives of domesticated crops contain many useful disease resistance (R) genes. Introducing this natural resistance is an elegant way of managing disease. However, traditional methods for introducing R genes typically involve long breeding trajectories to avoid linkage drag, i.e. the simultaneous introduction of deleterious traits. Furthermore, R genes tend to be overcome by the pathogen within a few seasons when deployed one at a time.

An approach to preventing a pathogen from quickly overcoming the resistance provided by a single R gene is to deploy simultaneously multiple R genes against the pathogen in a crop plant. Although such an approach can be accomplished by traditional plant breeding methods, the multiple R genes would very likely be found scattered throughout the genome of the plant of interest, making the combination of the multiple R genes into a single plant extremely laborious and time consuming. Alternatively, transgenic approaches can be used to rapidly deploy multiple R genes into a single crop plant. The multiple R genes can be introduced into a single crop plant as transgenes via routine genetic engineering techniques. Preferably, the multiple R genes would be introduced as a single, multi-transgene cassette that segregates as a single locus to facilitate the rapid transfer of the multiple R genes to breeding lines and crop plant cultivars.

Traditional map-based cloning of R genes, however, is still challenging. First, large tracts of plant genomes are inaccessible to map-based genetics due to lack of recombination. Second, most R genes belong to a structural class of genes called NB-LRRs, which tend to reside in complex clusters, and many hundreds of NB-LRRs populate a typical plant genome. The scientist therefore frequently delimits a map interval containing multiple NB-LRRs and must find out which confers the resistance of interest. Recently, a new method, which is known as Resistance Gene Enrichment Sequencing (RenSeq), has been reported that allows rapid scrutiny of all the NB-LRRs within a plant genome (i.e., the so-called “NB-LRRome”). (Jupe et al., 2013, Plant J. 76(3):530-44). While the RenSeq method can be used to the rapidly identify NB-LRR genes in a particular plant, the RenSeq method does not allow for the identification of an R gene that is specific to a plant disease of interest in the absence of additional map-based genetics approaches. Thus, a method for the rapid identification of an R gene for a particular disease of interest that does not depend on map-based genetics is desired to aid in the production of crop plants with multiple R genes directed to a particular pathogen.

### BRIEF SUMMARY OF THE INVENTION

In one aspect, the present invention provides methods for identifying a plant disease resistance (R) gene for a plant disease of interest. The methods involve obtaining at least one group of nucleic acids that are derived from a mutagenized plant that is susceptible to the plant disease of interest. The methods further involve selecting from the group of nucleic acids a subgroup of nucleic acids by hybridizing in solution the group of nucleic acids and a set of bait sequences to form a hybridization mixture. The bait sequences of the invention are designed to hybridize to one or more genes from at least one R gene family. The methods further involve isolating from the hybridization mixture the subgroup of nucleic acids that are hybridized to the bait sequences from any nucleic acids that are not hybridized to the bait sequences and sequencing the subgroup of nucleic acids to obtain a collection of nucleic acid sequences. The methods further involve comparing such nucleic acid sequences with corresponding sequences of one or more genes that are derived from a reference plant that is resistant to the plant disease of interest and then identifying at least one nucleic acid sequence derived from the mutagenized plant that is not identical in sequence to a corresponding sequence from the reference plant, wherein the corresponding sequence comprises a nucleic acid sequence of at least a portion of an R gene for the plant disease of interest. The methods can optionally comprise the step of producing the mutagenized plant by exposing a plant that is resistant to the plant disease of interest or part thereof to an effective amount of a mutagen and selecting at least one progeny plant that is susceptible to the plant disease of interest.

In some embodiments of the invention, the methods for identifying a plant R gene can further comprise obtaining the corresponding sequences from the reference plant essentially as described in the last paragraph but starting with a reference group of nucleic acids that are derived from the reference plant instead of the group of nucleic acids that are derived from the mutagenized plant. In particular, such methods involve selecting a reference subgroup of nucleic acids from a group of nucleic acids that are derived from the reference plant, isolating the reference subgroup of nucleic acids, and sequencing the subgroup of reference nucleic acids to obtain a reference group of nucleic acid sequences, wherein the reference group of nucleic acid sequences comprises the one or more corresponding sequences.

In another aspect, the present invention provides methods for identifying a gene associated with a phenotypic change for a trait of interest. The methods involve obtaining at least one group of nucleic acids that are derived from a mutagenized organism that comprises a phenotypic change for the trait of interest relative to the phenotype of the trait of interest for a reference organism. The methods further involve selecting from the group of nucleic acids a subgroup of nucleic acids by hybridizing in solution the group of nucleic acids and a set of bait sequences to form a hybridization mixture. The bait sequences of the invention are designed to hybridize to one or more genes within a group or family of genes in the reference organism. The methods further involve isolating from the hybridization mixture the subgroup of nucleic acids that are hybridized to the bait sequences from any nucleic acids that are not hybridized to the bait sequences and sequencing the subgroup of nucleic acids to obtain a collection of nucleic acid sequences. The methods further involve comparing such nucleic acid sequences with corresponding sequences of one or more genes that are derived from a reference organism and then identifying at least one nucleic acid sequence derived from the mutagenized organism that is not identical in sequence to a corresponding sequence from the reference organism, wherein the non-identical sequence comprises at least a portion of a nucleic acid sequence of a gene associated with the phenotypic change of interest. The methods can optionally comprise the step of producing the mutagenized organism by exposing an organism that has a first phenotype for a trait of interest and selecting at least one progeny organism that has a second phenotype for the trait of interest, wherein the second phenotype is distinguishable from the first phenotype. In certain embodiments of the invention, the methods comprise obtaining the corresponding sequences from the reference organism.

### BRIEF DESCRIPTION OF THE DRAWINGS

FIG. 1. Dotplot of the genomic locus of Sr33 (horizontal) with the three contigs of the wild-type assembly (vertical) that match with 100% identity.

FIG. 2. Schematic representation of Sr33 and the wild-type contigs. The horizontal red bar shows the genomic sequence of Sr33 with its two introns (red/white regions). The brown bars below show the contigs and the red vertical lines mark the positions of the known point mutations.

### DETAILED DESCRIPTION OF THE INVENTION

### Overview

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

In one aspect, the present invention provides methods for the rapid identification of R genes from plants. The methods find use in the identification of new R genes that can be incorporated into a crop plant to confer resistance to a plant disease of interest. Such new R genes are desired by plant breeders to aid in the development of new crop plant varieties with enhanced resistance to one or more plant diseases.

The methods of the present invention do not depend on map-based genetics for the identification of a plant R gene for a disease of interest. The methods of present invention involve the use of Resistance Gene Enrichment Sequencing (RenSeq) as described by Jupe et al. (2013, Plant J., 76(3):530-44 and supplementary materials), herein incorporated in its entirety by reference. As reported in Jupe et al., RenSeq allows for the rapid scrutiny of all the NB-LRRs genes within a plant genome.

While the methods disclosed herein were initially developed by the present inventors to aid in the rapid identification of NB-LRR-type R genes from plants, the inventors recognized that their methods can also be for used in identifying a gene associated with a phenotypic change for a trait of interest in plants as well as other organisms. Thus, the methods of the present invention are not limited to identifying only NB-LRR-type R genes but can be used to identify other types of R genes and also genes that are associated with a phenotypic change of interest. As disclosed hereinbelow, the present invention further provides methods for rapidly identifying genes that are associated with a phenotypic change of interest in plants and other organisms. Such methods comprise the use of mutagenized organisms and an enrichment sequencing approach that is similar to RenSeq but which allows for the enrichment of a gene family or a group of genes other than a plant NB-LRR gene family.

Non-limiting embodiments of the invention include, for example, the following embodiments.

1. A method for identifying a plant resistance (R) gene for a plant disease of interest, the method comprising:
(a) selecting a subgroup of nucleic acids comprising
   (i) hybridizing in solution a group of nucleic acids and a set of bait sequences to form a hybridization mixture, wherein the group of nucleic acids are derived from a mutagenized plant that is susceptible to the plant disease of interest and wherein the bait sequences are designed to hybridize to one or more genes from at least one R gene family, and
   (ii) isolating from the hybridization mixture a subgroup of the nucleic acids that are hybridized to the bait sequences from nucleic acids that are not hybridized to the bait sequences;
(b) sequencing the subgroup of nucleic acids to obtain a collection of nucleic acid sequences;

(c) comparing the nucleic acid sequences obtained in (b) with corresponding sequences of the one or more genes that are derived from a reference plant that is resistant to the plant disease of interest; and
(d) identifying at least one nucleic acid sequence derived from the mutagenized plant that is not identical in sequence to a corresponding sequence from the reference plant, wherein the corresponding sequence comprises a nucleic acid sequence of at least a portion of an R gene for the plant disease of interest.

2. The method of embodiment 1, further comprising performing steps (a)-(d) one of more additional times, wherein each additional time the group of nucleic acids is derived from a different mutagenized plant.

3. The method of embodiment 1 or 2, wherein the mutagenized plant or plants and the reference plant are in the same genus.

4. The method of any one of embodiments 1-3, wherein the mutagenized plant or plants and the reference plant are the same species.

5. The method of any one of embodiments 1-4, wherein the mutagenized plant or plants is/are produced by mutagenizing at least one plant of the same genotype as the reference plant.

6. The method of any one of embodiments 1-5, wherein the mutagenized plant or plants was/were produced by mutagenesis comprising exposing at least one plant to a chemical mutagen or radiation.

7. The method of embodiment 6, wherein the chemical mutagen is selected from the group consisting of ethyl methanesulfonate (EMS), di-epoxy-butane (DEB), and sodium azide.

8. The method of any one of embodiments 1-7, wherein the mutagenized plant or plants was/were produced by mutagenesis comprising exposing at least one plant that is resistant to the plant disease of interest to a mutagen and selecting at least one progeny plant that is susceptible to the plant disease of interest.

9. The method of any one of embodiments 1-7, further comprising selecting at least one mutagenized plant from a population of mutagenized plants that was produced by exposing plants that are resistant to the plant disease of interest to an effective amount of a mutagen, wherein selecting comprises screening the population for susceptibility to the plant disease of interest and identifying at least one at least one mutagenized plant that is susceptible to the plant disease of interest.

10. The method of any one of embodiments 1-7, further comprising producing at least one mutagenized plant by exposing plants that are resistant to the plant disease of interest to an effective amount of a mutagen to produce a population of mutagenized plants and selecting from the population at least one plant that is susceptible to the plant disease of interest.

11. The method of any one of embodiments 8-10, wherein the plant that is resistant to the plant disease of interest is selected from the group consisting of, the reference plant, a plant comprising the same genotype as the reference plant, a plant comprising the same species as the reference plant, and a plant comprising the same species as the reference plant.

12. The method of any one of embodiments 1-11, further comprising before step (c), obtaining the corresponding sequences by,
selecting a reference subgroup of nucleic acids comprising hybridizing in solution a reference group of nucleic acids and the set of bait sequences to form a reference hybridization mixture, wherein the reference group of nucleic acids are derived the reference plant,
isolating from the reference hybridization mixture a reference subgroup of nucleic acids that are hybridized to the bait sequences from the reference nucleic acids that are not hybridized to the bait sequences and/or from any non-hybridized bait sequences, and
sequencing the subgroup of reference nucleic acids to obtain a reference collection of nucleic acid sequences, wherein the reference collection of nucleic acid sequences comprises the one or more corresponding sequences.

13. The method of any one of embodiments 1-12, wherein at least one of the mutagenized plant and the reference plant is a crop plant or a non-domesticated plant in the same family as at least one crop plant.

14. The method of any one of embodiments 1-13, wherein at least one of the mutagenized plant and the reference plant is a crop plant or a non-domesticated plant in the same genus as at least one crop plant.

15. The method any one of embodiments 1-14, wherein at least one of the mutagenized plant and the reference plant is a crop plant or a non-domesticated plant in the same species as at least one crop plant.

16. The method of any one of embodiments 1-15, wherein the mutagenized plant and the reference plant are monocots.

17. The method of any one of embodiments 13-16, wherein the crop plant is selected from the group consisting of wheat, maize, rice, barley, rye, sorghum, oat, millet, onion, sugarcane, palm, and banana.

18. The method of any one of embodiments 1-15, wherein the mutagenized plant and the reference plant are dicots.

19. The method of any one of embodiments 13-15 and 18, wherein the crop plant is selected from the group consisting of potato, tomato, pepper (Capsicum annuum), tobacco, canola, cotton, soybean, peanut, alfalfa, sunflower, and safflower.

20. The method of any one of embodiments 1-19, wherein the group of nucleic acids comprises fragmented genomic DNA.

21. The method of any one of embodiments 12-20, wherein the reference group of nucleic acids comprises fragmented genomic DNA.

22. The method of any one of embodiments 1-19, wherein the group of nucleic acids comprises RNA or cDNA derived from RNA.

23. The method of any one of embodiments 12-19 and 22, wherein the reference group of nucleic acids comprises RNA or cDNA derived from RNA.

24. The method of any one of embodiments 1-23, wherein the R gene encodes an NB-LRR protein.

25. The method of any one of embodiments 1-24, wherein the sequencing is next-generation sequencing.

26. The method of any one of embodiments 1-25, wherein the isolating step of (a)(ii) comprises contacting the hybridization mixture with at least one molecule or particle that binds to or is capable of separating the set of bait sequences from the hybridization mixture, and separating the set of bait sequences from the hybridization mixture to isolate a subgroup of nucleic acids that hybridize to the bait sequences from the group of nucleic acids.

27. The method of any one of embodiments 12-26, wherein the isolating of the reference nucleic acids that are hybridized to the bait sequences comprises contacting the reference hybridization mixture with the molecule or particle that binds to or is capable of separating the set of bait sequences from the reference hybridization mixture, and separating the set of bait sequences from the reference hybridization mixture to isolate a subgroup of reference nucleic acids that hybridize to the bait sequences from the group of reference nucleic acids.

28. The method of any one of embodiments 1-27, wherein the bait sequences are polynucleotides between about 60 nucleotides and 180 nucleotides in length.

29. The method of any one of embodiments 1-28, wherein each of the bait sequences is designed to hybridize with a part of at least one member of the R gene family.

30. The method of any one of embodiments 1-29, wherein each of the bait sequences is designed to be at least 80% identical to a part of the coding region of a member of the R gene family.

31. The method of any one of embodiments 1-30, further comprising confirming that the R gene is capable of conferring resistance to the plant disease of interest to a susceptible plant.

32. The method of embodiment 31, wherein confirming comprises introducing the R gene for the plant disease of interest into a susceptible plant and exposing the susceptible plant to a pathogen that is the causal agent for the plant disease of interest under conditions favorable for development of the plant disease.

33. The method of embodiment 32, wherein introducing the R gene comprises transforming the susceptible plant with a nucleic acid molecule encoding the protein encoded by the R gene.

34. The method of embodiment 33, wherein introducing the R gene further comprises sexual reproduction.

35. The method of embodiment 34, wherein introducing the R gene does not comprise transforming the susceptible plant with a nucleic acid molecule encoding the protein encoded by the R gene.

36. The method of embodiment 35, wherein introducing the R gene comprises sexual reproduction.

37. A method for identifying a plant resistance (R) gene for a plant disease of interest, the method comprising:
(a) producing at least one mutagenized plant by exposing plants that are resistant to the plant disease of interest to an effective amount of a mutagen to produce a population of mutagenized plants and selecting from the population at least one plant that is susceptible to the plant disease of interest;
(b) selecting a subgroup of nucleic acids comprising
   (i) hybridizing in solution a group of nucleic acids and a set of bait sequences to form a hybridization mixture, wherein the group of nucleic acids are derived from the mutagenized plant and wherein the bait sequences are designed to hybridize to one or more genes from at least one R gene family, and
   (ii) isolating from the hybridization mixture the subgroup of the nucleic acids that are hybridized to the bait sequences from nucleic acids that are not hybridized to the bait sequences, wherein the bait sequences are designed to hybridize to one or more genes from at least one R gene family;
(c) sequencing the subgroup of nucleic acids to obtain a collection of nucleic acid sequences;
(d) comparing the nucleic acid sequences obtained in (c) with corresponding sequences of the one or more genes that are derived from a reference plant that is resistant to the plant disease of interest; and
(e) identifying at least one nucleic acid sequence derived from the mutagenized plant that is not identical in sequence to a corresponding sequence from the reference plant, wherein the corresponding sequence comprises a nucleic acid sequence of at least a portion of an R gene for the plant disease of interest.

38. The method of embodiment 37, further comprising performing steps (b)-(e) one of more additional times, wherein each additional time the group of nucleic acids is derived from a different mutagenized plant produced according to step (a).

39. The method of embodiment 37 or 38, wherein the mutagenized plant or plants and the reference plant are in the same genus.

40. The method of any one of embodiments 37-39, wherein the mutagenized plant or plants and the reference plant are the same species.

41. The method of any one of embodiments 36-40, wherein the plant that is resistant to the plant disease of interest is the reference plant or a plant comprising the same genotype as the reference plant.

42. The method of any one of embodiments 37-41, wherein the mutagen is a chemical mutagen.

43. The method of embodiment 42, wherein the chemical mutagen is selected from the group consisting of ethyl methanesulfonate (EMS), di-epoxy-butane (DEB), and sodium azide.

44. The method of any one of embodiments 37-43, further comprising before step (d), obtaining the corresponding sequences by,
selecting a reference subgroup of nucleic acids comprising hybridizing in solution a reference group of nucleic acids and the set of bait sequences to form a reference hybridization mixture, wherein the reference group of nucleic acids are derived the reference plant,
isolating from the reference hybridization mixture a reference subgroup of nucleic acids that are hybridized to the bait sequences from the reference nucleic acids that are not hybridized to the bait sequences and/or from any non-hybridized bait sequences, and
sequencing the subgroup of reference nucleic acids to obtain a reference collection of nucleic acid sequences, wherein the reference collection of nucleic acid sequences comprises the one or more corresponding sequences.

45. The method of any one of embodiments 37-44, wherein at least one of the mutagenized plant and the reference plant is a crop plant or a non-domesticated plant in the same family as at least one crop plant.

46. The method of any one of embodiments 37-45, wherein at least one of the mutagenized plant and the reference plant is a crop plant or a non-domesticated plant in the same genus as at least one crop plant.

47. The method any one of embodiments 37-46, wherein at least one of the mutagenized plant and the reference plant is a crop plant or a non-domesticated plant in the same species as at least one crop plant.

48. The method of any one of embodiments 37-47, wherein the mutagenized plant and the reference plant are monocots.

49. The method of any one of embodiments 45-48, wherein the crop plant is selected from the group consisting of wheat, maize, rice, barley, rye, sorghum, oat, millet, onion, sugarcane, palm, and banana.

50. The method of any one of embodiments 37-47, wherein the mutagenized plant and the reference plant are dicots.

51. The method of any one of embodiments 45-47 and 50, wherein the crop plant is selected from the group consisting of potato, tomato, pepper (Capsicum annuum), tobacco, canola, cotton, soybean, peanut, alfalfa, sunflower, and safflower.

52. The method of any one of embodiments 37-50, wherein the group of nucleic acids comprises fragmented genomic DNA.

53. The method of any one of embodiments 44-52, wherein the reference group of nucleic acids comprises fragmented genomic DNA.

54. The method of any one of embodiments 37-53, wherein the group of nucleic acids comprises RNA or cDNA derived from RNA.

55. The method of any one of embodiments 44-51 and 54, wherein the reference group of nucleic acids comprises RNA or cDNA derived from RNA.

56. The method of any one of embodiments 37-55, wherein the R gene encodes an NB-LRR protein.

57. The method of any one of embodiments 37-56, wherein the sequencing is next-generation sequencing.

58. The method of any one of embodiments 37-57, wherein the isolating step of (b)(ii) comprises contacting the hybridization mixture with at least one molecule or particle that binds to or is capable of separating the set of bait sequences from the hybridization mixture, and separating the set of bait sequences from the hybridization mixture to isolate a subgroup of nucleic acids that hybridize to the bait sequences from the group of nucleic acids.

59. The method of any one of embodiments 44-58, wherein the isolating of the reference nucleic acids that are hybridized to the bait sequences comprises contacting the reference hybridization mixture with the molecule or particle that binds to or is capable of separating the set of bait sequences from the reference hybridization mixture, and separating the set of bait sequences from the reference hybridization mixture to isolate a subgroup of reference nucleic acids that hybridize to the bait sequences from the group of reference nucleic acids.

60. The method of any one of embodiments 37-59, wherein the bait sequences are polynucleotides between about 60 nucleotides and 180 nucleotides in length.

61. The method of any one of embodiments 37-60, wherein each of the bait sequences is designed to hybridize with a part of at least one member of the R gene family.

62. The method of any one of embodiments 37-61, wherein each of the bait sequences is designed to be at least 80% identical to a part of the coding region of a member of the R gene family.

63. The method of any one of embodiments 37-62, further comprising confirming that the R gene is capable of conferring resistance to the plant disease of interest to a susceptible plant.

64. The method of embodiment 63, wherein confirming comprises introducing the R gene for the plant disease of interest into a susceptible plant and exposing the susceptible plant to a pathogen that is the causal agent for the plant disease of interest under conditions favorable for development of the plant disease.

65. The method of embodiment 64, wherein introducing the R gene comprises transforming the susceptible plant with a nucleic acid molecule encoding the protein encoded by the R gene.

66. The method of embodiment 65, wherein introducing the R gene further comprises sexual reproduction.

67. The method of embodiment 64, wherein introducing the R gene does not comprise transforming the susceptible plant with a nucleic acid molecule encoding the protein encoded by the R gene.

68. The method of embodiment 67, wherein introducing the R gene comprises sexual reproduction.

69. A method for identifying a gene associated with a phenotypic change for a trait of interest, the method comprising:
(a) selecting a subgroup of nucleic acids comprising
   (i) hybridizing in solution a group of nucleic acids and a set of bait sequences to form a hybridization mixture, wherein the group of nucleic acids are derived from a mutagenized organism that comprises a phenotypic change for the trait of interest relative to the phenotype of the trait of interest for a reference organism, wherein the phenotypic change is induced by mutagenesis, and wherein the bait sequences are designed to hybridize to genes from a group or family of genes in the reference organism, and
   (ii) isolating from the hybridization mixture a subgroup of nucleic acids that are hybridized to the bait sequences from any nucleic acids that are not hybridized to the bait sequences;
(b) sequencing the subgroup of nucleic acids to obtain a collection of nucleic acid sequences;
(c) comparing the nucleic acid sequences obtained in (b) with corresponding sequences of the group or family of genes that are derived from the reference organism; and
(d) identifying at least one nucleic acid sequence derived from the mutagenized organism that is not identical in sequence to a corresponding sequence from the reference organism, wherein the non-identical sequence comprises at least a portion of a nucleic acid sequence of a gene associated with a phenotypic change of interest.

70. The method of embodiment 69, further comprising performing steps (a)-(d) one of more additional times, wherein each additional time the group of nucleic acids is derived from a different mutagenized organism.

71. The method of embodiment 69 or 70, wherein the reference organism and the mutagenized organism(s) are eukaryotic organisms.

72. The method of embodiment 71, wherein the eukaryotic organisms are selected from the group consisting of plants, animals, fungi, algae, protozoans, and oomyctes.

73. The method of embodiment 72, wherein the animals are mammals.

74. The method of embodiment 73, wherein the mammals are humans.

75. The method of any one of embodiments 69-74, wherein the reference organism and the mutagenized organism(s) are selected from the group consisting in vitro-cultured human cells or in vitro-cultured human tissue.

76. The method of any one of embodiments 69-75, wherein the mutagenized organism or mutagenized organisms is/are produced by mutagenizing the reference organism or an organism of the same genotype as the reference organism.

77. The method of embodiment 69-76, wherein the mutagenized organism or mutagenized organisms was/were produced by mutagenesis comprising exposing at least one organism to a chemical mutagen or radiation.

78. The method of embodiment 77, wherein the chemical mutagen is selected from the group consisting of ethyl methanesulfonate (EMS), di-epoxy-butane (DEB), sodium azide, and N-ethyl-N-nitrosourea (ENU).

79. The method of any one of embodiments 69-78, further comprising producing at least one mutagenized organism by exposing organisms that comprise a first phenotype for a trait of interest to an effective amount of a mutagen to produce a population of mutagenized organisms and selecting from the population at least one organism that comprises a second phenotype for the trait of interest, wherein the second phenotype is distinguishable from the first phenotype, wherein the organism is not a human being.

80. The method of embodiment 79, wherein the organism that comprises the first phenotype is the reference organism or is the same genotype as the reference organism.

81. The method of any one of embodiments 69-80, further comprising before step (c), obtaining the corresponding sequences by,
selecting a reference subgroup of nucleic acids comprising hybridizing in solution a reference group of nucleic acids and the set of bait sequences to form a reference hybridization mixture, wherein the reference group of nucleic acids are derived the reference organism,
isolating from the reference hybridization mixture a reference subgroup of nucleic acids that are hybridized to the bait sequences from reference nucleic acids that are not hybridized to the bait sequences, and
sequencing the subgroup of reference nucleic acids to obtain a reference collection of nucleic acid sequences, wherein the reference collection of nucleic acid sequences comprises the one or more corresponding sequences.

82. The method of any one of embodiments 69-81, wherein the mutagenized organism(s) and the reference organism are in the same family.

83. The method of any one of embodiments 69-82, wherein the mutagenized organism(s) and the reference organism are from the same genus.

84. The method any one of embodiments 69-83, wherein the mutagenized organism(s) and the reference organism are the same species.

85. The method of any one of embodiments 69-84, wherein the group of nucleic acids comprises fragmented genomic DNA.

86. The method of any one of embodiments 81-85, wherein the reference group of nucleic acids comprises fragmented genomic DNA.

87. The method of any one of embodiments 69-84, wherein the group of nucleic acids comprises RNA or cDNA derived from RNA.

88. The method of any one of embodiments 81-84 and 87, wherein the reference group of nucleic acids comprises RNA or cDNA derived from RNA.

89. The method of any one of embodiments 69-88, wherein the sequencing is next-generation sequencing.

90. The method of any one of embodiments 69-89, wherein the isolating step of (a)(ii) comprises contacting the hybridization mixture with molecule or particle that binds to or is capable of separating the set of bait sequences from the hybridization mixture, and separating the set of bait sequences from the hybridization mixture to isolate a subgroup of nucleic acids that hybridize to the bait sequences from the group of nucleic acids.

91. The method of any one of embodiments 69-90, wherein the isolating of the reference nucleic acids that are hybridized to the bait sequences comprises contacting the reference hybridization mixture with molecule or particle that binds to or is capable of separating the set of bait sequences from the reference hybridization mixture, and separating the set of bait sequences from the reference hybridization mixture to isolate a subgroup of reference nucleic acids that hybridize to the bait sequences from the group of reference nucleic acids.

92. The method of any one of embodiments 69-91, wherein the bait sequences are polynucleotides between about 60 nucleotides and 180 nucleotides in length.

93. The method of any one of embodiments 67-92, wherein one or more of the bait sequences are designed to hybridize specifically to a conserved region in one or more of the genes in the group or family of genes.

94. The method of any one of embodiments 67-93, further comprising confirming that the gene associated with the phenotypic change of interest of part (d) is associated with the phenotypic change.

95. The method of any one of embodiments 67-94, wherein the mutagenized organism is a plant, the gene associated with the trait of interest is an R gene, wherein the trait of interest is resistance of a plant disease of interest, and the phenotypic change is from resistance to the disease of interest to susceptibility to the disease of interest.

96. The method of embodiment 95, wherein the R gene encodes an NB-LRR protein.

Additional embodiments of the invention are discussed in detail below.

### DEFINITIONS

In the context of this disclosure, a number of terms are used. The following definitions are provided immediately below. Other definitions can be found throughout the disclosure.

The methods of the present invention involve the use of plants and other organisms. Thus, for the present invention, the term “plant” is understood to mean a whole plant or any part thereof, unless noted otherwise or apparent from the context of use. As used herein, the term “plant” also includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Likewise, the term “organism” is understood to mean a whole organism or any part thereof including, for example, a cell, unless noted otherwise or apparent from the context of use. Furthermore, it is to be understood that the terms “plant” and “organism” further encompass in vitro-cultured tissues and in vitro-cultured cells of a plant and an organism, respectively.

In the description of the various methods of present invention, singular terms are used to describe the plants and organisms that are used in the disclosed methods. Such terms include, but are not limited to, susceptible plant, mutagenized plant, reference plant, resistant plant, progeny, mutagenized organism, and reference organism. However, the use of such singular terms is not intended to limit the methods to the use of, for example, only a single plant or organism. Thus, as used herein, the terms “plant”, “progeny plant”, and “progeny” encompasses a single plant or two or more plants or a portion of a plant such as, for example, a plant organ or organs, or one or more plant cells, unless stated otherwise or apparent for the context of use. Likewise, the term “organism”, “progeny organism”, and “progeny” encompasses a single organism or two or more organisms or a portion of an organism such as, for example, a limb or limbs, or one or more cells, unless stated otherwise or apparent for the context of use.

The present invention can involve the use of two or more plants or organisms that have the “same genotype”. For the present invention, “same genotype” is intended to mean that the two or more plants or organisms are characterized by having essentially identical genomes, unless noted otherwise or apparent from the context of use. In other words, the two or more plants or the two or more organisms are isogenic.

As used herein, an “isogenic line” is comprised of plants or other organisms that have the same genotype.

Certain embodiments of the present invention can comprise the use of non-domesticated plants. A “non-domesticated” plant is a plant that has not been subjected to human selection or has otherwise been genetically modified by humans. Usually, non-domesticated plants are collected from an uncultivated area or are non-selected, non-genetically-modified descendants of a plant or plants collected from an uncultivated area. It is recognized that non-domesticated relatives of crop plants are often used as sources of new R genes that are not found in the genomes of their domesticated relatives. Typically, a non-domesticated relative of a crop plant that is used as a source of a new R gene belongs to the same species as the crop plant or belongs to a different species that is in the same genus as the genus of the crop plant. Occasionally, a non-domesticated relative of a crop plant that is used as a source of a new R gene belongs to a species and genus that are different from the crop plant but belong to the same family as the crop plant.

As used herein, “progeny” refers to a descendant or descendants of any subsequent generation of a plant or other organism of the present invention unless noted otherwise or apparent from the context of use. For example, the first, second, third, and fourth generation descendants of a particular plant or organism of the present invention are progeny of that particular plant or organism, respectively.

The methods of the present involve the use of a “mutagenized plant” or “mutagenized organism”. These terms are intended to encompass not only the initially produced mutagenized plant or mutagenized organism (M₁) but also progeny of the initially produced, mutagenized plant or mutagenized organism, respectively. In the methods disclosed herein wherein a phenotypic change is due to a recessive mutation, the phenotypic change will generally not be detectable in the plant or organism that is exposed to the mutagen. Progeny of the plant or organism comprising the phenotypic change of interest (e.g. change from resistance to susceptibility to a disease of interest) will typically be used as the mutagenized plant or mutagenized organism, particularly progeny that display the desired phenotype that was induced by the mutagenesis. For plants with perfect flowers or both male and female flowers on the same individual plant, the first-generation progeny plants (M₂) of the initially produced mutagenized plant (M₁) are preferably produced by selfing the initially produced mutagenized plant.

As used herein, a “trait of interest” is any inherited characteristic of an organism that is genetically determined. Traits of interest include both qualitative and quantitative traits such as, for example, the presence of a particular metabolite, the level of a particular metabolite, resistance to a disease, resistance to an insect, resistance to a chemical (e.g., resistance to a plant to a herbicide), agricultural yield, color of an organism or any part thereof (e.g., eye color, hair color, leaf color, seed color, and flower color), and the like.

As used herein, “group of nucleic acids” means nucleic acids that are derived from a mutagenized plant or mutagenized organism that contain target sequences and are hybridized to bait sequences to select the target sequences.

As used herein, “group of reference nucleic acids” means nucleic acids that are derived from a reference plant or reference organism that contain target sequences and are hybridized to bait sequences to select the target sequences.

As used herein, “target sequences” are the set of sequences that one desires to isolate from the group of nucleic acids. In some preferred embodiments of the invention, the target sequences are R gene sequences. In other embodiments, the target sequences are nucleic acid sequences from one or more genes, particularly genes within a gene family or other group of genes.

As used herein, a “bait sequence” is a nucleic acid molecule comprising a nucleotide sequence that is designed to hybridize to a target sequence. A “target sequence” is at least a portion of a gene of interest. It is recognized that a bait sequence can comprise additional nucleotides beyond those nucleotides that are intended to hybridize to the complement of the target sequence. For the purposes of determining percent nucleotide sequence identity between a bait sequence and a target sequence, only the portion of “bait sequence” that is designed to hybridize to a target sequence will be used for determining percent nucleotide sequence identity between a bait sequence and a target sequence unless stated otherwise.

As used herein, a “corresponding sequence” is a sequence derived from a reference plant or reference organism that corresponds to a nucleic acid derived from the reference plant or reference organism, respectively. For example, a corresponding sequence can be the sequence of all or a part of an R gene from the reference plant, which corresponds to the same R gene or part thereof in the mutagenized plant. It is recognized that a nucleotide sequence of a nucleic acid derived from a mutagenized plant or mutagenized organism is not identical to its corresponding sequence when the nucleic acid derived from a mutagenized plant or mutagenized organism comprises a mutation.

As used herein, an “effective amount of a mutagen” is an amount of the mutagen that causes the desired level of mutations in the plant or other organism after a certain period of exposure to the mutagen. Generally, there is not a single effective amount of mutagen that is capable of causing the desired level of mutations. Instead, any amount of the mutagen within a certain range of amounts will be capable of producing the desired level of mutations in the plant or other organism. Such an effective amount of a mutagen can be, for example, a certain concentration of a mutagen in an aqueous solution when the plant or organism is exposed to the mutagen in an aqueous solution. Alternatively, an effective amount of a mutagen can be a particular dosage of radiation. It is recognized that the effective amount of a mutagen will often be empirically determined because the effective amount can vary depending on any number of factors including, for example, the mutagen used, the age of the plant or organism, the duration of the exposure to the mutagen, the desired level of induced mutations, the temperature during the exposure, the composition of the solution comprising the mutagen, and the like. It is further recognized that methods of mutagenizing plants and other organized are known in the art and that a person of skill in the art will know or know how to determine the effective amount of a mutagen to achieve the expected level of mutations for a particular plant or organism of interest.

In addition to the terms defined above, additional terms related to the present are defined throughout the disclosure.

### DESCRIPTION

In one aspect, the present invention provides methods for identifying a plant R gene for a plant disease of interest. The methods involve obtaining a group of nucleic acids derived from a mutagenized plant that is susceptible to a plant disease of interest and then selecting a subgroup of nucleic acids by hybridizing in a solution the group of nucleic acids and a set of bait sequences that are designed to hybridize to one or more genes from at least one R gene family.

In some embodiments, the methods can additionally comprise selecting one or more mutagenized plants from a population of mutagenized plants that was produced by exposing plants that are resistant to the plant disease of interest to an effective amount of a mutagen. While the present invention does not depend on a particular method for selecting the desired mutagenized plant or plants, generally selecting the mutagenized plant or plants will comprise screening the population of mutagenized plants for susceptibility to the plant disease of interest and identifying at least one mutagenized plant that is susceptible to the plant disease of interest. The methods of the present invention do not depend on a particular method of screening a population of mutagenized plants for susceptibility to a plant disease of interest. Any screening method known in the art can be used in the methods of the present invention.

Generally, screening the population of mutagenized plants for susceptibility to the plant disease of interest involves growing the population of mutagenized plants for a period of time under field, greenhouse, or controlled-environment (e.g., growth chamber) conditions that are favorable for the development of the disease of interest and in the presence of the pathogen that is the causal agent of the disease of interest. Such screening further involves assessing the plants for disease severity one or more times during the period of time and/or at the end of the period of time. Typically, the plants will be inoculated at the beginning of the screening with a sufficient amount of the pathogen for the development of disease symptoms during the period of time utilized. Those of skill in the art will know the sufficient amount of a pathogen of interest, or know how to determine such a sufficient amount, so as to ensure that disease symptoms will develop in a susceptible plant during the period of time utilized. Disease severity can be assessed, for example, using standard methods for the disease of interest that are known in the art.

In some other embodiments, the methods can additionally comprise producing the mutagenized plant or plants by exposing plants that are resistant to the plant disease of interest to an effective amount of a mutagen to produce a population of mutagenized organisms and selecting from the population at least one plant that is susceptible to the plant disease of interest as described above. Generally, such selecting comprises screening a population of mutagenized plants for susceptibility to the plant disease of interest and identifying at least one, but preferably more than one, mutagenized plant that is susceptible to the plant disease of interest as described above. In certain embodiments, the mutagen is a chemical mutagen, preferably methanesulfonate (EMS). EMS is a known mutagen that typically induces G/C-to-A/T transitions in DNA (Jander et al. (2003) Plant Physiol. 131:139-146). In other embodiments, the chemical mutagen is di-epoxy-butane (DEB), which has been reported to yield a complementary spectrum of single nucleotide mutations when compared to EMS (Malinovsky et al., 2010, PLoS One. 5(9):e12586). However, the present invention is not limited to mutagenizing a plant with EMS, DEB, or other chemical mutagen. Any mutagenesis method known in the art can be used to produce the plants and other organisms of the present invention. Such mutagenesis methods can involve, for example, the use of any one or more of the following mutagens: radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 2500 to 2900 nm), and chemical mutagens such as sodium azide, base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones, N-ethyl-N-nitrosourea), azide, hydroxylamine, nitrous acid, or acridines. Further details of mutagenesis of plants and mutation breeding can be found in “Principals of Cultivar Development” Fehr, 1993 Macmillan Publishing Company the disclosure of which is incorporated herein by reference. In one embodiment of the invention, the mutagenized plants are produced as described in Periyannan et al. (2013), Science 341:786-788 and supplementary materials; herein incorporated by reference.

The methods for identifying a plant R gene for a plant disease of interest further comprise hybridizing in solution a group of nucleic acids derived from a mutagenized plant and a set of bait sequences to form a hybridization mixture. In some embodiments of the invention, the bait sequences are designed to hybridize to one or more genes from at least one R gene family in a plant or a group of closely related plants, such as, for example, plants from two or more species in the same genus or even plants from two or more species within the same plant family. For example, a set of bait sequences for NB-LRR genes can be designed using the predicted NB-LRR genes from the sequenced genomes of two or more of the following species in Triticeae: barley (Hordeum vulgare), hexaploid bread wheat (Triticum aestivum), tetraploid pasta wheat (T. durum), T. urartu, Aegilops tauschii, and Aegilops sharonensis. In a preferred embodiment of the invention, a set of bait sequences of NB-LRR genes is designed using the predicted NB-LRR genes from the sequenced genomes of all six of aforementioned species in Triticeae. In another embodiment of the In a preferred embodiment of the invention, a set of bait sequences of NB-LRR genes is designed using the predicted NB-LRR genes from the sequenced genomes of one, two, three, or more of the following species in the Poaceae family (also known as the Gramineae or grass family): Brachypodium distachyon, maize (Zea mays), sorghum (Sorghum bicolor), barley (Hordeum vulgare), hexaploid bread wheat (Triticum aestivum), tetraploid pasta wheat (T. durum), T. urartu, Aegilops tauschii, and Aegilops sharonensis. In a preferred embodiment of the invention, a set of bait sequences of NB-LRR genes is designed using the predicted NB-LRR genes from the sequenced genomes of all nine of aforementioned species in Poaceae.

The bait sequences of the present invention are designed to hybridize to genes or parts thereof, preferably to the coding regions of genes or parts thereof. The initial step in designing bait sequences is to select a gene family or group of genes, such as, for example, an R gene family. Source sequences for designing the bait sequences can be obtained from any species that is known to possess one or more members of the gene family and for which the gene sequences are known or otherwise can be obtained by methods involved sequencing genomic or cDNA from the species of interest. For example, the source sequences for designing the bait sequences can be obtained from the same species as the reference plant and/or one or more additional species that are in the same family and/or genus as the reference plant. Alternatively, the source sequences for designing the bait sequences can be obtained from any one or more species of interest. In preferred embodiments of the invention, the source sequences for designing the bait sequences are obtained from the same species as the reference plant, and optionally one or more additional species within the same family and/or genus as the reference plant. It is recognized that bait sequences that are designed using source sequences derived from the same species as the reference plant or the mutagenized plant are more likely to hybridize and capture target sequences derived from the reference plant or the mutagenized plant, respectively, than bait sequences that are designed using source sequences from another species. While it is preferable that all or at least some of the source sequences of genes in the gene family of interest (e.g. R gene family) are known for a particular species, source sequences can be obtained from any species of interest by, for example, whole-genome sequencing or any other method described elsewhere herein or otherwise known in the art.

It is also recognized that a bait sequence does need to be identical to a particular target sequence or its complement to hybridize to the target sequence in the methods disclosed herein. While the present invention is not bound by any particular mechanism, it is believed that bait sequences that comprise at least about 70%, 75%, or 80% nucleotide sequence identity to a target sequence can be used in the methods of the present invention to capture the target sequence. Preferably, a bait sequence of the present invention comprises at least 80%, 85%, 90%, 95%, or a 100% nucleotide sequence identity to a target sequence. Because a bait sequence of the present invention can contain one or more additional non-target-specific nucleotides on its 5′ end and/or 3′ end, percent identity between is bait sequence is determined using only the entire target-specific portion of a bait sequence and the full-length target sequence, unless stated otherwise or apparent from the context of usage. Such a target-specific region of a bait sequence is that region of the bait sequence is designed to hybridize to a target sequence of a gene. Moreover, it is understood due to the complementary nature or nucleic acid molecules that any reference herein to a nucleotide sequence encompasses both the nucleotide sequence (sense sequence) and its full-length complement or complementary sequence (anti-sense sequence). For example, a reference to “a bait sequence that hybridizes to a target sequence comprising 100% sequence identity to the bait sequence” is understood to mean that the bait sequence hybridizes to the complement of a target sequence comprising 100% nucleotide sequence identity to the bait sequence.

While it is preferable to have identified intron/exon boundaries in the genes of interest before designing bait sequences, such intron/exon boundaries may not be known at the time the bait sequences are designed. Bait sequences that comprise intron sequences and/or intron/exon boundaries might not be effective at capturing target sequences since introns are less conserved among different species. However, in a set of bait sequences comprising, for example, 60,000 bait sequences, the inclusion of some bait sequences that are incapable of capturing target sequences in a group of nucleic acids are derived from a mutagenized plant and/or a reference plant is not expected to have a significant detrimental effect on the methods of the present invention.

In one embodiment of the invention, bait sequence are designed to hybridize to target sequences in NB-LRR genes. To identity NB-LRR containing genes, protein sequences are scanned for NB-ARC domains using pfam_scan, version 1.5 (Finn et al., 2008, Nuc. Acids Res. 36:D281-D288). If protein sequences are not available, nucleotide sequences are translated into their six open reading frames and all six sequences are scanned. Once source sequences are identified, bait sequences can be produced. However, depending on the circumstances, it may be desirable to reduce the number of source sequences by, for example, eliminating or reducing redundancy. In one approach, redundancy can be eliminated or reduced using the program CD-Hit, which is a widely used program for clustering and comparing protein or nucleotide sequences (Fu et al., 2012, Bioinformatics, 28:3150-3152). Alternatively, an iterative approach can be used in which all source sequences are aligned to each other. Whenever a bait is generated, the bait's motif is masked in the remaining source sequences. Lowering the threshold of identity percentage for both CD-Hit approach and the iterative pipeline can reduce the resulting number of baits, but might reduce the chance to capture a target sequence. Another approach that can be used to alter the number of bait sequences in a set of bait sequences is to adjust the coverage of the source sequences by the bait sequences by varying tiling from, for example, 0.5- to 4-fold tiling.

In preferred embodiments of the invention, it is desirable to avoid producing bait sequences that hybridize with other bait sequences. Therefore, before the bait sequence polynucleotides are produced, each potential bait sequences can be aligned by reverse complementary with all other potential bait sequences. It is recognized that whenever a potential bait sequence aligns with another potential bait sequence, one of the two potential bait sequences is then synthesized as its reverse complementary polynucleotide.

The methods for identifying an R gene for a plant disease of interest comprise selecting a subgroup of nucleic acids by first hybridizing in solution a group of nucleic acids derived from a mutagenized plant that is susceptible to the plant disease of interest and a set of bait sequences designed to hybridize to one or more genes from at least one R gene family to form a hybridization mixture and then isolating from the hybridization mixture a subgroup of the nucleic acids that are hybridized to the bait sequences from nucleic acids that are not hybridized to the bait sequences.

The use of such bait sequences to select and isolate a subgroup of nucleic acids from a group of nucleic acids has been previously described in U.S. Pat. App. Pub. No. 20100029498 and Gnirke et al. (2009, Nat. Biotechnol. 27(2): 182-189), both of which are herein incorporated by reference. In general, the sequence composition of the set of bait sequences determines the subgroup of nucleic acids directly selected from the group of nucleic acids and further that the subgroup of nucleic acids is a part or all of a set of target sequences that is desired to be selected. In a preferred embodiment of the present invention, the subgroup of nucleic acids are selected using the MYbaits target enrichment system according the manufacturer's directions (Mycroarray, Ann Arbor, Mich., USA) with bait sequences designed to hybridize to one or more genes from at least one R gene family as disclosed elsewhere herein. In other embodiments of the present invention, the subgroup of nucleic acids are selected using the SureSelect target enrichment system (Agilent Technologies, Santa Clara, Calif., USA), the TruSelect exome enrichment system (Illumina, Inc., San Diego, Calif., USA), or the NimbleGen target enrichment system (Roche NimbleGen, Inc., Madison, Wis., USA) according the manufacturer's directions with bait sequences designed to hybridize to one or more genes from at least one R gene family as disclosed elsewhere herein.

To aid in separating the subgroup of the nucleic acids that are hybridized to the bait sequences from nucleic acids that are not hybridized to the bait sequences, the bait sequences of the present invention can comprise an affinity tag on each bait sequence, particularly an affinity tag including, but limited to, biotin molecules, magnetic particles, haptens, or other tag molecules that permit isolation of molecules tagged with the tag molecule. The subgroup of the nucleic acids that are hybridized to the bait sequences can then be separated from the bait sequences using routine methods known in the art for separating the strands of double-stranded nucleic acids. See, for example, U.S. Pat. App. Pub. No. 20100029498.

In certain embodiments, the methods further comprise subjecting the isolated subgroup of nucleic acids to one or more additional rounds of solution hybridization with the same or a different set of bait sequences that are designed to hybridize to one or more genes from the R gene family. For example, a first set of bait sequences can be designed to hybridize to certain conserved regions in the R gene family and a second set of bait sequences can be designed to hybridize to conserved regions in the R gene family but is not identical to the first set of bait sequences. The second set of bait sequences can, for example, be designed to hybridize to a subset of the conserved regions of the first subset, the same conserved regions as the first subset and one or more additional conserved regions, or different conserved regions than the first subset. Alternatively, the second set of bait sequences can be designed to hybridize to the same conserved regions of the first subset but is comprised of one or more bait sequences that are not identical in sequence to any sequence found in the first subset of bait sequences.

Following separation of the subgroup of nucleic acids from the bait sequences, the methods of the invention comprises sequencing the subgroup of nucleic acids are to obtain a collection of nucleic acid sequences. As used herein, “sequencing” refers to sequencing methods for determining the order of nucleotides in a nucleic acid molecule, particularly DNA. It is understood that “sequencing the subgroup of nucleic acids” does not require the sequencing of all of the individual nucleic acids in the subgroup of nucleic acids. It is further understood that “sequencing the subgroup of nucleic acids” does not require that full-length sequences be obtained for each individual nucleic acid. In preferred embodiments of the invention, the methods of the present invention comprise sequencing most or all of the individual nucleic acids in the subgroup of nucleic acids whereby the sequences individual nucleic acids are partial or full-length sequences.

Any DNA sequencing method known in the art can be used in the methods provided herein. Non-limiting examples of DNA sequencing methods useful in the methods provided herein include, for example, the next-generation sequencing technologies as described in Egan et al. (2012) Am. J. of Bot. 99(2):175-185, herein incorporated by reference. The phrase “next-generation sequencing” or NGS refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization. In particular embodiments, the DNA fragment library is sequenced using the Illumina MiSeq system (Illumina, Inc., San Diego, Calif., USA), Illumina HiSeq 2000 system (Illumina, Inc., San Diego, Calif., USA), Illumina HiSeq 2500 system (Illumina, Inc., San Diego, Calif., USA), or the PacBio RS II system (Pacific Biosciences of California, Inc., Menlo Park, Calif., USA).

Sequencing of the subgroup of nucleic acids will result in a collection of individual sequences corresponding to individual nucleic acids in the subgroup of nucleic acids. As used herein, the term “read” refers to the sequence of a DNA fragment obtained after sequencing. In some embodiments, sequencing produces about 500,000, about 1 million, about 1.5 million, about 2 million, about 2.5 million, about 3 million, or about 5 million reads from the DNA sequence library. In certain embodiments, the reads are paired-end reads, wherein the DNA fragment is sequenced from both ends of the molecule. Depending on size of an individual nucleic acid, the paired-end reads can result in the full-length sequence of the individual nucleic acid whereby there is an overlapping region of sequence of the paired-end reads. Typically, however, the paired-end reads will not overlap and the sequence obtained for an individual nucleic acid will be less than full-length.

In some embodiments of the invention involving the use of next-generation sequencing, the sequencing information obtained from sequencing of the subgroup of nucleic acids will be analyzed and assembled into the sequences of the individual nucleic acids in the subgroup of nucleic acids using computer software such as, for example, CLC Assembly Cell v. 4.2.0 (CLC bio, Cambridge, Mass., USA), Velvet (Birney, 2008, Genome Res. 18(5):821-29), ABySS Simpson et al., 2009, Genome Res. 19(6):1117-1123), Allpath L G (Gnerre et al., 2011, PNAS 108(4):1513-1518, MSR-CA (Zimin et al., 2013, Bioinformatics. 29(21):2669-2677), or MIRA (available on the worldwide web at sourceforge.net/projects/mira-assembler).

The methods of the invention further comprise comparing the nucleic acid sequences of the subgroup of nucleic acids derived from the mutagenized plant with the corresponding sequences of the one or more genes that are derived from a reference plant that is resistant to the plant disease of interest and identifying at least one nucleic acid sequence derived from the mutagenized plant that is not identical in sequence to a corresponding sequence from the reference plant. Preferably, the non-identical corresponding sequence comprises a nucleic acid sequence of at least a portion of an R gene for the plant disease of interest.

Generally, the identification of a nucleic acid sequence derived from the mutagenized plant that is not identical in sequence to a corresponding sequence comprises an integrated comparison of sequences derived from the mutagenized plant to sequences derived from the reference plant (e.g. wild-type) assembly. Any method known in the art for comparing sequences from the mutagenized plant to the sequences of the reference plant can be used in the methods of the present invention. For example, in a first step, raw sequence data from an individual mutagenized plant is mapped against contigs from the reference plant and the result is converted to a base-centric view (pileup) in which bwa (version 0.7.4) (Li and Durbin, 2009, Bioinformatics, 25(14):1754-1760) is used for mapping and SAMtools (version 0.1.19) (Li et al., 2009, Bioinformatics, 25(16):2078-2079) for pileup. Other mapping software can also be used such as, for example, bowtie Langmead et al., 2009, Genome Biol. 10(3):R25).

In a preferred embodiment of the invention involving use of a mutagenized plant produced by EMS mutagenesis, the identification of the one nucleic acid sequence derived from the mutagenized plant that is not identical in sequence to a corresponding sequence from the reference plant can be implemented as a Java program (available on the worldwide web at java.com). The identification of single nucleotide polymorphisms (SNPs) is included in this embodiment of the methods of the present invention but the identification of SNPs can also be implemented using additional external software, such as, for example, SAMtools (version 0.1.19) (Li et al., 2009, Bioinformatics, 25(16):2078-2079) or GATK (McKenna et al., 2010, Genome Res. 20(9):1297-1303).

In preferred embodiments of the invention, a high sensitivity for identifying SNPs rather than specificity for target sequences is desirable. To cope with off-target sequences in the reference (e.g. wild-type) plant sequence assembly, subsequences of the contigs are classified as either NB-LRR-like or off-target/non-coding. Only SNPs or deletions within NB-LRR like subsequences are regarded for further target identification. The classification is based on an alignment of known NB-LRR sequences to the wild-type contigs. The usage of the bait-sequences as known NB-LRR sequences is sufficient. However, it is preferred to use NB-LRR protein sequences of closely related species if available. Any software to perform local alignments between sequences can be used for this step. For example, NCBI BLAST (version 2.2.28+) (available on the worldwide web at ncbi.nlm.nih.gov; Zhang et al., 2000, J. Comput. Biol. 7(1-2):203-214) can be used to perform the local alignments.

In some embodiments of the invention, two, three, four or more mutagenized plants are used, wherein a group of nucleic acids is derived from each mutagenized plant. In such embodiments, a subgroup of nucleic acid is selected, sequenced, and compared to corresponding sequences of the reference plant for each additional mutagenized plant as described above for a first mutagenized plant. In such embodiments, the SNPs and deletions are recorded for each contig from each of the mutagenized plants. In a preferred embodiment, an SNP can be defined, for example, by a certain reference allele frequency (the base represented by the wild-type contig) of, for example, less than 10% and a minimum coverage of a fourth of the mean coverage over all NB-LRR-like subsequences. A deletion can be defined as a stretch of bases in an NB-LRR-like subsequence that has a coverage of, for example, less than 10% of the overall mean coverage. Generally, an SNP that is present in nucleic acid sequence derived from more than one mutagenized plant is likely to be an artifact caused by an error in the wild-type assembly (i.e. assembly of the sequence from the reference plant) or unspecific mapping rather than by the same mutation in two mutagenized plants. Preferably, only those SNPs and/or deletions that are present in a nucleic acid derived from a single mutagenized plant are regarded for further analysis.

Occasionally, suboptimal wild-type sequence assemblies can hinder the identification of a target gene (e.g. R gene). A well-known difficulty in a de novo assembly concerns collapsed contigs. Very similar regions (e.g. repeats, gene families) within a genome might be combined into one consensus sequence during the assembly. For example, this might happen for NB-LRR genes in the wild-type assembly. However, it is recognized that there a number of methods known in the art for dealing with collapsed contigs and that the present invention does not depend on a particular for dealing with collapsed contigs. For example, more investment can be made into generating a better quality wild-type assembly to avoid collapsed contigs, e.g. using long read technologies or mate pair libraries. Alternatively or additionally, the allele frequency in a mapping of wild-type reads against the wild-type assembly can be compared to the allele frequency in the mapping of mutant-line reads against the wild-type assembly. A significant difference would reveal the NB-LRR region as a candidate. Subsequently, the collapsed contig can be resolved by localized assemblies.

Another potential difficulty resulting from suboptimal wild-type sequence assemblies is a fragmented wild-type assembly. For example, if the wild-type assembly is fragmented, or if an intron in the target gene is larger than the length of the captured fragments, different parts of the target gene can be on different contigs. However, it is recognized that there a number of methods known in the art for dealing with fragmented wild-type assemblies and that the present invention does not depend on a particular for dealing with fragmented wild-type assemblies. For example, more investment can be made into generating a better quality wild-type assembly, e.g. using long read technologies or mate pair libraries. Alternatively, it can be desirable to use additional mutagenized plants in the method of the present invention whereby a nucleic acid sequence is obtained from at least one mutagenized plant that comprises at least one part of the R gene without being fragmented.

In certain embodiments of the present invention, the mutagenized plant is produced by mutagenizing a plant that is resistant to the plant disease of interest. Preferably, such a resistant plant and the reference plant are the same species or are from different species within the same genus. More preferably, the resistant plant and the reference plant are the same species and genotype, and in some embodiments, the mutagenized plant is produced by mutagenizing the reference plant.

In some embodiments, the methods for identifying a plant R gene for a plant disease of interest can further comprise hybridizing in solution the bait sequences essentially as described above but using a reference group of nucleic acids that are derived the reference plant instead of the group of nucleic acids that are derived from the mutagenized plant. In particular, the methods comprise selecting a reference subgroup of nucleic acids by hybridizing in solution a reference group of nucleic acids and the set of bait sequences to form a reference hybridization mixture and then isolating from the reference hybridization mixture a reference subgroup of nucleic acids that are hybridized to the bait sequences from the reference nucleic acids that are not hybridized to the bait sequences and/or from any non-hybridized bait sequences. The methods further comprise sequencing the subgroup of reference nucleic acids essentially as described above to obtain a reference collection of nucleic acid sequences, wherein the reference collection of nucleic acid sequences comprises the one or more corresponding sequences.

The methods for identifying a plant R gene for a plant disease of interest can be used with any plant including, for example, crop plants and non-domesticated plants. In a preferred embodiment of the invention, the mutagenized plant is produced by mutagenizing a non-domesticated relative of a crop plant that is resistant to the plant disease of interest. It is recognized that such non-domesticated relatives of crop plants can often be the source of new R genes, which might not be present in the genome of a crop plant of interest. The non-domesticated relative of a crop plant can be a species within the same family as the crop plant, more preferably a species within the same genus as the crop plant, most preferably the same species as the crop plant. Often, a non-domesticated relative is obtained from the wild, particularly from a center of origin or center of diversity for the crop plant species.

Plants of interest include, for example, both monocot and dicot plants, preferably monocot and dicot crop plants. Preferred monocot crop plants include, but are not limited to, wheat, maize, rice, barley, rye, sorghum, oat, millet, onion, sugarcane, palm, and banana. Preferred dicot crop plants include, but are not limited to, potato, tomato, pepper (Capsicum annuum), tobacco, canola, cotton, soybean, peanut, alfalfa, sunflower, and safflower. Other plants of interest include, for example, fruit trees such as apple, pear, plum, and citrus (e.g. sweet orange, sour orange, blood orange, mandarin orange, lemon, lime, grapefruit, and kumquat).

In another aspect, the present invention provides methods for identifying a gene associated with a phenotypic change for a trait of interest. Preferably, the phenotypic change results for an induced mutation in a single gene or locus. The methods involve obtaining at least one group of nucleic acids that are derived from a mutagenized organism that comprises a phenotypic change for the trait of interest relative to the phenotype of the trait of interest for a reference organism. The methods can be used with any organism of interest that is capable of being mutagenized to produce the desired phenotypic change for the trait of interest. Preferred organisms are eukaryotic organisms such as, for example, plants, animals, fungi, algae, protozoa, and oomycetes.

In some embodiments, the methods can additionally comprise selecting the mutagenized organism from a population of mutagenized organism that was produced by exposing organisms that comprise a first phenotype for a trait of interest to an effective amount of a mutagen and selecting at least one progeny organism that comprises a second phenotype for the trait of interest, wherein the second phenotype is distinguishable from the first phenotype. The desired phenotypic change in the trait of interest is the change from the first phenotype to the second phenotype in at least one organism following mutagenesis. While the present invention does not depend on a particular method for selecting the desired mutagenized organism or organisms, generally selecting the mutagenized organism or organisms will comprise screening the population of mutagenized organism for the second phenotype for the trait of interest and identifying at least one mutagenized organism that comprises the second phenotype. The methods of the present invention do not depend on a particular method of screening a population of mutagenized organisms for the second phenotype. Any screening method known in the art can be used in the methods of the present invention.

In some other embodiments, the methods can additionally comprise producing the mutagenized organism by exposing organisms that comprise the first phenotype for the trait of interest to an effective amount of a mutagen to produce a population of mutagenized organisms and selecting from the population at least one organism that comprises the second phenotype for the trait of interest as described above. Generally, such selecting comprises screening the population of mutagenized organisms for the second phenotype and identifying at least one mutagenized organism that comprises the second phenotype as described above. In certain embodiments, the mutagen is a chemical mutagen, preferably ethyl methanesulfonate (EMS), di-epoxy-butane (DEB), sodium azide, or N-ethyl-N-nitrosourea (ENU). However, the present invention is not limited to mutagenizing an organism with a chemical mutagen or to any particular mutagenesis method. An organism of the present invention can be mutagenized using any one or more of the mutagens described above. Mutagenesis protocols are known in the art for organisms of interest. See, for example, Salinger, A. P. and Justice, M. J., “Mouse Mutagenesis Using N-Ethyl-N-Nitrosourea (ENU),” CSH Protocols, 2008, 3(4):1-5; herein incorporated by reference.

The methods for identifying a gene associated with a phenotypic change for a trait of interest comprise selecting a subgroup of nucleic acids by first hybridizing in solution a group of nucleic acids derived from a mutagenized organism and a set of bait sequences designed to hybridize to one or more genes within a group or family of genes in the reference organism to form a hybridization mixture and then isolating from the hybridization mixture the subgroup of nucleic acids that are hybridized to the bait sequences from any nucleic acids that are not hybridized to the bait sequences. The use of bait sequences is described above. In certain embodiments, the methods can further comprise subjecting the isolated subgroup of nucleic acids to one or more additional rounds of solution hybridization with the same or a different set of bait sequences essentially as described above but using bait sequences designed to hybridize to one or more genes within a group or family of genes in the reference organism.

Following separation of the subgroup of nucleic acids from the bait sequences, the methods for identifying a gene associated with a phenotypic change for a trait of interest comprise sequencing the subgroup of nucleic acids to obtain a collection of nucleic acid sequences and comparing the nucleic acid sequences of the subgroup of nucleic acids derived from the mutagenized organism with the corresponding sequences of the one or more genes that are derived from a reference organism and identifying at least one nucleic acid sequence derived from the mutagenized organism that is not identical in sequence to a corresponding sequence from the reference organism as described above.

In some embodiments, the methods can further comprise hybridizing in solution the bait sequences essentially as described above but using a reference group of nucleic acids that are derived the reference organism instead of the group of nucleic acids that are derived from the mutagenized organism. In particular, the methods comprise selecting a reference subgroup of nucleic acids by hybridizing in solution a reference group of nucleic acids and the set of bait sequences to form a reference hybridization mixture and then isolating from the reference hybridization mixture a reference subgroup of nucleic acids that are hybridized to the bait sequences from the reference nucleic acids that are not hybridized to the bait sequences and/or from any non-hybridized bait sequences. The methods further comprise sequencing the subgroup of reference nucleic acids essentially as described above to obtain a reference collection of nucleic acid sequences, wherein the reference collection of nucleic acid sequences comprises the one or more corresponding sequences.

The group of nucleic acids and/or group of reference nucleic acids in some embodiments is fragmented genomic DNA. Genomic DNA may be fragmented by physical shearing methods, enzymatic cleavage methods, chemical cleavage methods, and other methods well known to those skilled in the art. It is recognized that the optimal average size of the fragmented genomic DNA will depend on a number of factors including, for example, the particular target enrichment system used, the average size of the bait sequences, and/or the DNA sequencing method. In preferred embodiments, the fragmented genomic DNA will be at least about 300 bp in size. If desired, the fragmented DNA can be size selected by any of the standard methods known in the art. The group of nucleic acids typically contains all or substantially all of the complexity of the genome. The term “substantially all” in this context refers to the possibility that there may in practice be some unwanted loss of genome complexity during the initial steps of the procedure. However, the methods described herein also are useful in cases where the group of nucleic acids is a portion of the genome, i.e., where the complexity of the genome is reduced by design. In such embodiments, the practitioner may use any selected portion of the genome with the methods described herein.

In some other embodiments, the group of nucleic acids and/or group of reference nucleic acids is RNA or cDNA derived from RNA. Methods for isolating RNA from plants and other organisms and for making cDNA from RNA are known in the art and/or described elsewhere herein. Generally, methods for making cDNA from RNA involve the use of reverse transcriptase and/or PCR amplification.

A bait sequence of the present invention is designed to hybridize specifically to the complements of one or more target sequences (e.g., R gene sequences). Generally, a bait sequence comprises at least about 60%, 65%, 70%, 75%, 80%, 90%, 95%, 96%, 97%, 98%, 99%, or 100% nucleotide sequence identity to each of the one or more target sequences.

The subgroup of nucleic acids, while ideally containing 100% of the target sequences (i.e., when the selection method selects all of the target sequences from the group of nucleic acids) and no additional non-targeted sequences, typically contains less than all of the target sequences and contains some amount of background of unwanted sequences. For example, more typically the subgroup of nucleic acids is at least about 20%, 30%, 40%, 50%, 60%, 70%, 75%, 80%, 85%, 90%, 95%, 98%, 99% or more of the target sequences. The purity of the subgroup (percentage of reads that align to the targets) is typically at least about 20%, 30%, 40%, 50%, 60%, 70%, 75%, 80%, 85%, 90%, 95%, 98%, 99% or more.

It is preferred that the bait sequences be tagged with an affinity tag. As noted above, preferably there is an affinity tag on each bait sequence in a set of bait sequences. Affinity tags include biotin molecules, magnetic particles, haptens, or other tag molecules that permit isolation of molecules tagged with the tag molecule. To incorporate a biotin molecule as an affinity tag, for example, the bait polynucleotides can be reamplified using one or more biotinylated primers in a reamplification process such as PCR.

As noted above, in some embodiments, the bait sequences are polynucleotides that are between about 40 nucleotides and about 400 nucleotides in length, more preferably between about 60 nucleotides and about 180 nucleotides in length, more preferably between about 80 nucleotides and about 120 nucleotides in length. In some embodiments, the target-specific sequences in the polynucleotides are between about 40 and about 400 nucleotides in length, more preferably between about 60 and about 180 nucleotides in length, more preferably between about 80 and about 120 nucleotides in length. Intermediate lengths in addition to those mentioned above also can be used in the methods of the invention, such as polynucleotides of about 40, 50, 60, 70, 80, 90, 100, 110, 120, 140, 160, 180, 200, 250, 300, 350, and 400 nucleotides in length, as well as polynucleotides of lengths between the above-mentioned lengths.

The number of bait sequences in a set of bait sequences can vary depending on a number of factors including, for example, the number of members in the R gene family, the sequence identity between the various members of the R gene family, the average length of the bait sequences, and the particular target enrichment system that is used for selecting the subgroup of nucleic acids. Generally, the number of bait sequences in a set of bait sequences is sufficient to hybridize to the entirety of the members of an R gene family (e.g. the NB-LRR gene family) of interest in the genome of the reference plant. It is recognized that the number of baits in a set of bait sequences can range from hundreds, to thousands, to tens of thousands, to hundreds of thousands, or more baits. It is recognized that target enrichment kits can be purchased with various numbers of baits custom designed for a particular target of interest (e.g. an R gene family). For example, MYbaits kits (Mycroarray, Ann Arbor, Mich., USA) are commercially available with 20,000, 40,000, 60,000 and 200,000 baits that are 120 mers (i.e. polynucleotides comprising 120 nucleotides).

RNA molecules preferably are used as bait sequences. A RNA-DNA duplex is more stable than a DNA-DNA duplex, and therefore provides for potentially better capture of nucleic acids. RNA bait sequences can be synthesized using any method known in the art. In some embodiments, in vitro transcription is used, for example based on adding RNA polymerase promoter sequences to one end of oligonucleotides. As is well known in the art, RNA promoter sequences can also be introduced during PCR amplification of bait sequences out of genomic DNA by tailing one primer of each target-specific primer pairs with an RNA-promoter sequence. If RNA is synthesized using biotinylated UTP, single stranded biotin-labeled RNA bait molecules are produced. In preferred embodiments, the RNA baits correspond to only one strand of the double-stranded DNA target. As those skilled in the art will appreciate, such RNA baits are not self-complementary and are therefore more effective as hybridization drivers. In certain embodiments, RNase-resistant RNA molecules are synthesized. Such molecules and their synthesis are well known in the art.

The present invention provides methods for identifying a plant R gene for a plant disease of interest. Such an R gene is capable of conferring upon a plant resistance to the plant disease of interest. Generally, when a plant comprising such an R gene is inoculated with the pathogen that causes the disease of interest, the severity of the disease is lower than the disease severity in a similarly inoculated control plant that lacks a functional form of the R gene. For the present invention such a control plant that lacks a functional form of the R gene is a susceptible plant for the particular disease of interest unless stated otherwise or apparent from the context of use. In certain embodiments of the invention, the severity of the disease is at least about 10%, 20%, 30%, 40%, 50%, 60%, 70%, 80%, 90%, or essentially 100% lower in a plant comprising the R gene than in a control plant lacking a functional form of the R gene. It is recognized that a mutagenized plant of the present invention will typically comprise a mutation in an R gene for a particular disease of interest that renders the R gene non-functional, whereby the mutagenized plant is susceptible to the plant disease of interest.

The methods for identifying a plant R gene for a plant disease of interest comprise the use of bait sequences that are designed to hybridize to one or more target genes from at least one R gene family. The term “R gene family” is used to refer to a family of structurally related genes, from a single plant species or two or more plant species in the same genus or same family. Typically, such an R gene family is comprised of at least one gene that is known to confer resistance to a plant disease of interest on a plant comprising the R gene. However, such an R gene family can also be comprised of one or more structurally related genes that do not, or are not known, to confer resistance to any plant disease. For the present invention, an R gene family or other gene family will be typically comprised of at least two genes but often more genes. Preferably, an R gene family or other gene family comprises about 10, 20, 30, 40, 50, 75, 100, 150, 200, 250, or more genes. In some embodiments, the R gene family is an NB-LRR-type R gene family and typically comprises at least about 100 genes. For example, the NB-LRR-type R gene family has been reported to comprise 319 genes for soybean, 200 genes for Arabidopsis thaliana, 398 genes for poplar, 600 genes for rice, 61 genes for cucumber, and 55 genes for papaya (Huang et al. (2009) Nature Genetics 41:1275-1281.

R gene families of interest for the present invention include, but are not limited to, R gene families comprising genes encoding receptor like proteins (RLPs), R gene families comprising genes encoding receptor like-protein kinases (RLKs), R gene families comprising genes encoding coiled-coiled protein kinases and R gene families comprising genes encoding NB-LRRs with various ‘decorations’ such as kinases and WRKY transcription factors. See, Yue et al. (2012, New Phytol. 193:1049-1063) for examples of various NB-LRR decorations.

The methods of the present invention can be used with nucleic acids derived from eukaryotic and prokaryotic organisms including, for example, plants, animals, fungi, oomyctes, alage, and bacteria. Examples of plant species of interest include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Peryea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Fruit trees and related plants include, for example, apples, pears, peaches, plums, oranges, grapefruits, limes, pomelos, palms, and bananas,

In specific embodiments, plants of the present invention are crop plants such as, for example, soybean, cotton, alfalfa, sunflower, canola (Brassica spp., particularly Brassica napus, Brassica rapa, Brassica juncea), safflower, peanut, sugarcane, maize (corn) sorghum, rice, wheat, millet, barley, triticale, tobacco, potato, tomato, pepper (Capsicum annuum).

Pathogens of the invention are bacteria, insects, nematodes, fungi, oomycetes, and parasitic plants such as Striga sp. and Orabanche sp. Specific pathogens for major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassicicola, Pythium ultimum, Peronospora parasitica, Fusarium roseum, Alternaria alternata; Alfalfa: Clavibacter michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Verticillium albo-atrum, Xanthomonas campestris p.v. alfalfae, Aphanomyces euteiches, Stemphylium herbarum, Stemphylium alfalfae, Colletotrichum trifolii, Leptosphaerulina briosiana, Uromyces striatus, Sclerotinia trifoliorum, Stagonospora meliloti, Stemphylium botryosum, Leptotrichila medicaginis; Wheat: Pseudomonas syringae p.v. atrofaciens, Urocystis agropyri, Xanthomonas campestris p.v. translucens, Pseudomonas syringae p.v. syringae, Alternaria alternate, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis fisp. tritici, Puccinia recondite f.sp. tritici, Puccinia striiformis, Pyrenophora tritici-repentis, Septoria nodorum, Zymoseptoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Plasmopora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Colletotrichum graminicola, Fusarium moniliforme var. subglutinans, Erwinia stewartii, F. verticillioides, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, C. sublineolum, Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinate, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.; Tomato: Corynebacterium michiganense pv. michiganense, Pseudomonas syringae pv. tomato, Ralstonia solanacearum, Xanthomonas vesicatoria, Xanthomonas perforans, Alternaria solani, Alternaria porri, Collectotrichum spp., Fulvia fulva Syn. Cladosporium fulvum, Fusarium oxysporum f. lycopersici, Leveillula taurica/Oidiopsis taurica, Phytophthora infestans, other Phytophthora spp., Pseudocercospora fuligena Syn. Cercospora fuligena, Sclerotium rolfsii, Septoria lycopersici, Meloidogyne spp.; Potato: Ralstonia solanacearum, Pseudomonas solanacearum, Erwinia carotovora subsp. Atroseptica Erwinia carotovora subsp. Carotovora, Pectobacterium carotovorum subsp. Atrosepticum, Pseudomonas fluorescens, Clavibacter michiganensis subsp. Sepedonicus, Corynebacterium sepedonicum, Streptomyces scabiei, Colletotrichum coccodes, Alternaria alternate, Mycovellosiella concors, Cercospora solani, Macrophomina phaseolina, Sclerotium bataticola, Choanephora cucurbitarum, Puccinia pittieriana, Aecidium cantensis, Alternaria solani, Fusarium spp., Phoma solanicola f. foveata, Botrytis cinerea, Botryotinia fuckeliana, Phytophthora infestans, Pythium spp., Phoma andigena var. andina, Pleospora herbarum, Stemphylium herbarum, Erysiphe cichoracearum, Spongospora subterranean Rhizoctonia solani, Thanatephorus cucumeris, Rosellinia sp. Dematophora sp., Septoria lycopersici, Helminthosporium solani, Polyscytalum pustulans, Sclerotium rolfsii, Athelia Angiosorus solani, Ulocladium atrum, Verticillium albo-atrum, V. dahlia, Synchytrium endobioticum, Sclerotinia sclerotiorum; Banana: Colletotrichum musae, Armillaria mellea, Armillaria tabescens, Pseudomonas solanacearum, Phyllachora musicola, Mycosphaerella fijiensis, Rosellinia bunodes, Pseudomas spp., Pestalotiopsis leprogena, Cercospora hayi, Pseudomonas solanacearum, Ceratocystis paradoxa, Verticillium theobromae, Trachysphaera fructigena, Cladosporium musae, Junghuhnia vincta, Cordana johnstonii, Cordana musae, Fusarium pallidoroseum, Colletotrichum musae, Verticillium theobromae, Fusarium spp., Acremonium spp., Cylindrocladium spp., Deightoniella torulosa, Nattrassia mangiferae, Dreschslera gigantean, Guignardia musae, Botryosphaeria ribis, Fusarium solani, Nectria haematococca, Fusarium oxysporum, Rhizoctonia spp., Colletotrichum musae, Uredo musae, Uromyces musae, Acrodontium simplex, Curvularia eragrostidis, Drechslera musae-sapientum, Leptosphaeria musarum, Pestalotiopsis disseminate, Ceratocystis paradoxa, Haplobasidion musae, Marasmiellus inoderma, Pseudomonas solanacearum, Radopholus similis, Lasiodiplodia theobromae, Fusarium pallidoroseum, Verticillium theobromae, Pestalotiopsis palmarum, Phaeoseptoria musae, Pyricularia grisea, Fusarium moniliforme, Gibberella fujikuroi, Erwinia carotovora, Erwinia chrysanthemi, Cylindrocarpon musae, Meloidogyne arenaria, Meloidogyne incognita, Meloidogyne javanica, Pratylenchus coffeae, Pratylenchus goodeyi, Pratylenchus brachyurus, Pratylenchus reniformia, Sclerotinia sclerotiorum, Nectria foliicola, Mycosphaerella musicola, Pseudocercospora musae, Limacinula tenuis, Mycosphaerella musae, Helicotylenchus multicinctus, Helicotylenchus dihystera, Nigrospora sphaerica, Trachysphaera frutigena, Ramichloridium musae, Verticillium theobromae.

Fungal pathogens include, but are not limited to, Colletotrichum graminocola, Diplodia maydis, Fusarium graminearum, and Fusarium verticillioides.

Bacterial pathogens include, but are not limited to, Agrobacterium tumefaciens, Candidatus Liberibacter asiaticus, Clavibacter michiganensis, Clavibacter sepedonicus, Dickeya dadantii, Dickeya solani, Erwinia amylovora, Pectobacterium atrosepticum, Pectobacterium carotovorum, Pseudomonas andropogonis, Pseudomonas avenae, Pseudomonas alboprecipitans, Pseudomonas fluorescens, Pseudomonas savastanoi, Pseudomonas solanacearum, Pseudomonas syringae, Ralstonia solanacearum, Xanthomonas axonopodis, Xanthomonas campestris, Xanthomonas citri, Xanthomonas perforans, Xanthomonas vesicatoria, Xanthomonas oryzae, and Xylella fastidiosa

Oomycete pathogens include, but are not limited to, Phytophthora infestans, Phytophthora ipomoeae, Phytophthora mirabilis, Phytophthora phaseoli, Phytophthora megasperma fsp. glycinea, Phytophthora megasperma, and Phytophthora cryptogea.

Viruses include any plant virus, for example, tobacco or cucumber mosaic virus, ringspot virus, necrosis virus, maize dwarf mosaic virus, etc.

Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

The methods of the invention can involve introducing into a plant of interest an R gene or other gene identified by the methods disclosed herein. For example, an R gene identified by the methods of the present invention can be introduced into a susceptible plant or part thereof to confirm that the R gene confers resistance upon the plant to the plant disease of interest. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptide gain access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

An R gene identified by the methods of the present invention can be introduced by stable or transient transformation into a susceptible plant or part thereof to confirm that the R gene confers resistance upon the plant to the plant disease of interest. Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

In specific embodiments, the R gene or other identified gene of the invention can be provided to a plant using a variety of transient transformation methods. For example, an R gene identified by the methods of the present invention can be introduced by transient transformation into a susceptible plant or part thereof to confirm that the R gene confers resistance upon the plant to the plant disease of interest. Such transient transformation methods include, but are not limited to, the introduction of the gene thereof directly into the plant or the introduction of the corresponding transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference.

In other embodiments, a gene of the invention may be introduced into plants by contacting plants with a virus or viral nucleic acids. Generally, such methods involve incorporating a nucleotide construct of the invention within a viral DNA or RNA molecule. It is recognized that a gene of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a gene or nucleic acid construct at a specific location in the genome of a plant or other organism. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site, which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome. Other methods for targeted insertion of a gene or nucleic acid construct at a specific location in the genome of a plant or other organism include, for example, those involving fusion proteins with a nuclease domain and engineered DNA-binding domain such as a transcription activator-like effector (TAL) or zinc-finger protein DNA-binding domain. See, for example, WO 2010/079430, WO 2010/079430; WO 2011/072246; Townsend et al. (2009) Nature 459:442-445; Shukla et al. (2009) Nature 459, 437-441; Bibikova et al. (2003) Science 300, 764; Urnov et al. (2005) Nature 435, 646; Wright et al. (2005) The Plant Journal 44:693-705; and U.S. Pat. Nos. 7,163,824 and 7,001,768, all of which are herein incorporated by reference in their entireties.

For the methods of the present invention, various changes in phenotype are of interest in plants and other organisms. For plants, changes in phenotype of interest include, for example, modifying the fatty acid composition in a plant, altering the amino acid content of a plant, altering a plant's pathogen defense mechanism, and the like.

Genes of interest for crop plants are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. Genes of interest include, generally, those involved in oil, starch, carbohydrate, or nutrient metabolism as well as those affecting seed size, sucrose loading, and the like.

R genes and other plant genes identified herein can be stacked in plants with genes for other traits. Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the GAT gene; see, for example, U.S. Publication No. 20040082770 and WO 03/092360); or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

In some embodiments of the invention, the methods involve the use of nucleic acids derived from one or more mutagenized animals and optionally involve producing a mutagenized animal by exposing an animal to a mutagen. In some embodiments, the mutagenized animals are mammals including, for example, mice, rats, and in vitro-cultured human cells and/or in vitro-cultured human tissues. The present inventors neither condone nor claim methods involving the production of mutagenized human beings. It is understood that the term “mutagenized human” encompasses mutagenized in vitro-cultured human cells and/or in vitro-cultured human tissues but specifically excludes mutagenized human beings.

The methods of the present invention involve the use of nucleic acids that are derived from a plant or other organism. Such derived nucleic acids include, for example, DNA and RNA. Methods for isolating nucleic acids from plants and other organisms are disclosed elsewhere herein or otherwise known in the art. In certain embodiments, the nucleic acids can be isolated from one or more plants or other organisms and used in the methods disclosed herein without further amplification and/or modification. In other embodiments, the nucleic acids can be isolated and then amplified by, for example, polymerase chain reaction (PCR) amplification using methods disclosed elsewhere herein or otherwise known in the art and/or modified. The nucleic acids can be modified after isolation by methods known in art including, for example, reverse transcription of isolated RNA into cDNA, attaching or incorporating a detectable label and the like. Additionally, the isolated nucleic acids can be amplified before being modified.

In a PCR approach, oligonucleotide primers can be designed for use in PCR reactions to amplify corresponding DNA sequences from, for example, cDNA or genomic DNA derived from any plant or other organism of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those skilled in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et at (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The use of the term “nucleic acids” is not intended to limit the present invention to nucleic acids comprising DNA. Those of ordinary skill in the art will recognize that nucleic acids can comprise ribonucleotides and combinations of ribonucleotides and deoxyribonucleotides. Such deoxyribonucleotides and ribonucleotides include both naturally occurring molecules (e.g., DNA and RNA) and synthetic analogues. The nucleic acids of the invention also encompass all forms of sequences including, but not limited to, single-stranded forms, double-stranded forms, hairpins, stem-and-loop structures, and the like.

The nucleic acids of the invention can be provided in expression cassettes for expression in the plant or other organism of interest. The cassette will include 5′ and 3′ regulatory sequences operably linked to a gene or coding sequence of a gene identified by the methods disclosed herein. “Operably linked” is intended to mean a functional linkage between two or more elements. For example, an operable linkage between a nucleic acid of interest and a regulatory sequence (i.e., a promoter) is functional link that allows for expression of nucleic acid of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the nucleic acid to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a nucleic acid of the invention, and a transcriptional and translational termination region (i.e., termination region) functional in a plant or other organism of interest. The regulatory regions (i.e., promoters, transcriptional regulatory regions, and translational termination regions) and/or the nucleic acid of the invention may be native/analogous to the host cell or to each other. Alternatively, the regulatory regions and/or the nucleic acid of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous nucleic acid is from a species different from the species from which the polynucleotide was derived, or, if from the same/analogous species, one or both are substantially modified from their original form and/or genomic locus, or the promoter is not the native promoter for the operably linked nucleic acid. While it may be optimal to express the sequences using heterologous promoters, the native promoter sequences may be used.

The termination region may be native with the transcriptional initiation region, may be native with the operably linked nucleic acid of interest, may be native with the plant host, or may be derived from another source (i.e., foreign or heterologous) to the promoter, the nucleic acid of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the nucleic acids may be optimized for increased expression in the transformed plant. That is, the nucleic acids can be synthesized using plant-preferred codons for improved expression in plant. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders for use in plants are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome and host plant or other organism. The nucleic acids can be combined with constitutive, tissue-preferred, or other promoters for expression in plants and other organisms.]

Such constitutive promoters for use in plants include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611. Pathogen-inducible promoters for use in plants include, for example, those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used in the present invention.

The following examples are offered by way of illustration and not by way of limitation.

### Example 1

### Cereal NB-LRR Capture

### Source Sequences

Potential source sequences for the cereal NB-LRR RNA bait library are all publicly available genome and transcriptome sequences. It has been shown that sequences from related species are already sufficient to capture NB-LRR sequences (Jupe et al, 2013). Current public resources for Triticeae are listed in Table 1.

TABLE 1
Public Sequence Resources for Triticeae.*

| Species | Data Type | PubMed ID | Ref. |
|---|---|---|---|
| Triticum aestivum | Genomic | 23192148 | Brenchley et al., 2012, |
| | | | Nature 491:705-710 |
| Hordeum vulgare | Genomic | 23075845 | Mayer et al., 2012, |
| | | | Nature 491:711-716 |
| Aegilops tauschii | Genomic | 23535592 | Jia et al., 2013, |
| | | | Nature 496:91-95 |
| Triticum urartu | Transcriptome | 23800085 | Krasileva et al., 2013, |
| | | | Genome Biol. 14(6):R66 |
| Triticum durum | Transcriptome | 23800085 | Krasileva et al., 2013, |
| | | | Genome Biol. 14(6):R66 |

*See Nature (2012) 491(7426):705-10; herein incorporated by reference.

### Method to Identify NB-LRR Sequences

The baits for the library have a length of 120 bp and will theoretically capture fragments with an identity of 80% (Jupe et al., 2013). A “wrong” bait sequence might not hybidise with any fragment. A large amount of baits will increase the cost of the bait library. A “wrong” bait sequence might also capture a sequence that is not part of a NB-LRR gene. This will reduce the coverage (read depth) of target sequences.

Data sets come in different complexity and quality. To identity NB-LRR containing genes, protein sequences are scanned for NB-ARC domains using pfam_scan, version 1.5. If protein sequences are not available, nucleotide sequences are translated into their six open reading frames and all six sequences are scanned.
1. The optimal case is a sequenced genome with readily predicted genes. This includes protein sequences and a gff-file to link protein sequences to genomic exons. The protein sequences can be scanned for NB-ARC domains and the baits can be generated from the resulting genomic sequence.
2. Transcript assemblies without genomic support are a good source for baits. However, exon junctions are unknown and might produce “wrong” baits. Either the open reading frame of the transcript can be detected or the entire transcript is translated into the six possible reading frames and each translated protein sequence is then scanned for NB-ARC domains.

Additional steps for the generation of a bait library are minimizing of redundancies in source NB-LRR sequences to reduce costs for bait libraries and, as a precaution, the scanning for repetitive sequences in the source NB-LRR sequences, which should be avoided. To reduce redundancy, source sequences are clustered using cd-hit. The identity threshold depends on the amount of source sequences gathered in the steps above and the number of baits. Repeat-masking can be done using RepeatMasker. For Triticeae, a good repeat library is the ITMI Triticeae Repeat Sequence Database (TREP).

Finally, reverse complementary baits have to be avoided. By aligning all source sequences against themselves, reverse complementary sequences can be identified and reversed.

### Command Line Calls for Programs Mentioned Above

pfam_scan.pl source_genomes/genes.protein.fasta-dir data/pfam/>result.pfam.txt cd-hit-est -i resultSequences1.fasta -o resultSequences_cdhit80.fasta -T 16 -M 16000 -c 0.8 RepeatMasker -lib ˜/sequenceCapture/TREP.fasta resultSequences_cdhit90.fasta

### Pilot Study Sr33

Sr33 is a wheat stem rust resistance gene that has been cloned and published. One of the final aids in the identification of Sr33 included the sequencing of an allelic series of EMS-induced Sr33 mutants (Table 2). We used RenSeq on those mutant lines to re-clone Sr33. It should be noted that the cloned sequence of Sr33 was not included in the generation of the bait library.

TABLE 2
EMS-Induced sr33 Mutants (Periyannan et al. 2013)

| | | | | Refer- | |
|---|---|---|---|---|---|
| Sr33 | | | Posi- | ence | Mutant |
| mutant | Type | Accession number | tion | allele | allele |
| E6 | point | gi\|522700529\|gb\|KF031302.1\| | 2147 | C | T |
| | mutant | | | | |
| E7 | point | gi\|522700527\|gb\|KF031301.1\| | 752 | C | T |
| | mutant | | | | |
| E8 | point | gi\|522700531\|gb\|KF031303.1\| | 2311 | C | T |
| | mutant | | | | |
| E9 | point | gi\|522700525\|gb\|KF031300.1\| | 746 | G | A |
| | mutant | | | | |
| E2 | (large) | N/A | N/A | N/A | N/A |
| | deletion | | | | |
| | mutant | | | | |
| E3 | (large) | N/A | N/A | N/A | N/A |
| | deletion | | | | |
| | mutant | | | | |
| E4 | deletion | N/A | N/A | N/A | N/A |
| | mutant | | | | |
| E5 | deletion | N/A | N/A | N/A | N/A |
| | mutant | | | | |

### Sequencing

In a pilot experiment, the wild-type wheat parent, and mutants E7, E9 and E5 were sequenced. Two samples were multiplexed on each of two lanes of a 250 bp paired-end (PE) MiSeq run (Table 3).

TABLE 3
Sequencing of Sr33 mutants.

| Tissue | Library | Run |
|---|---|---|
| Wild Type | LIB5750 | 1 |
| E5 | LIB5751 | 1 |
| E9 | LIB6017 | 2 |
| E7 | LIB6018 | 2 |

### Wild-Type Assembly

Wild-type data was assembled using CLC Assembly Cell v. 4.2.0. Assembly statistics are shown in Table 4.

TABLE 4
Assembly statistics for the captured NB-LRRs from
the wild-type Sr33 parent.

| | Number | Sum | | | Number of |
|---|---|---|---|---|---|
| | of | length | Longest | N50 | contigs > |
| File name | contigs | of contigs | contig | length | 1 kb |
| pilot1_wt.fasta | 408,744 | 174,950,913 | 79,998 | 451 | 10,506 |

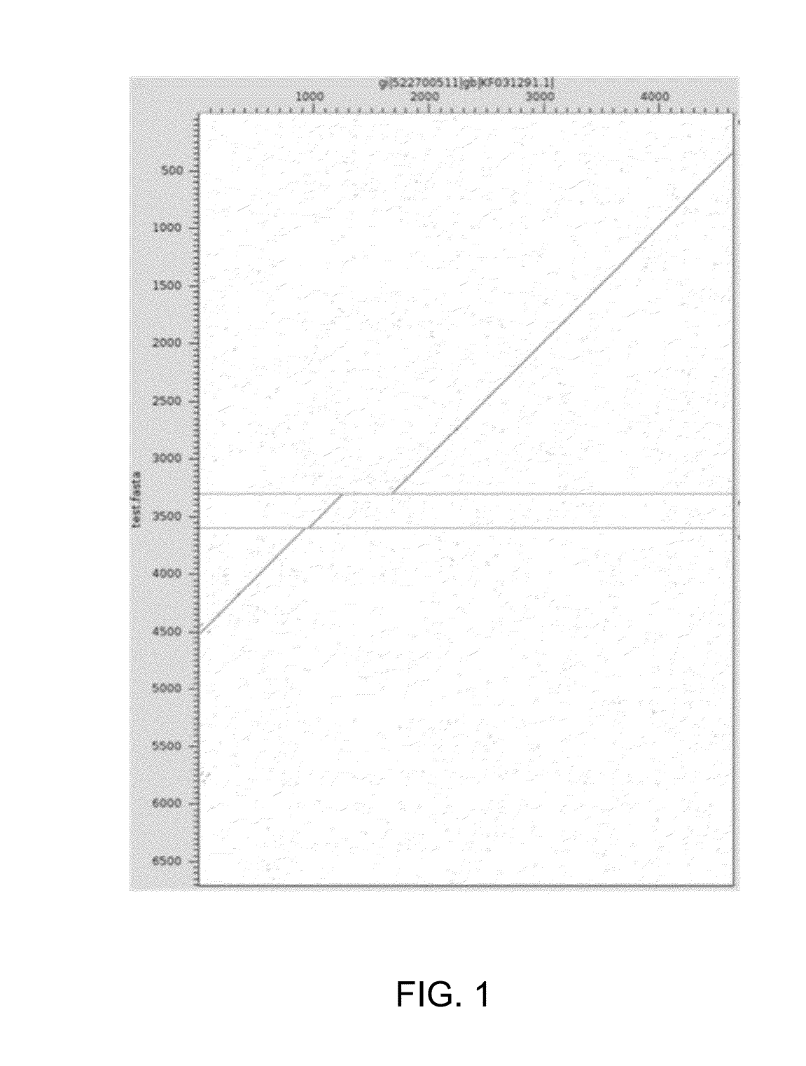

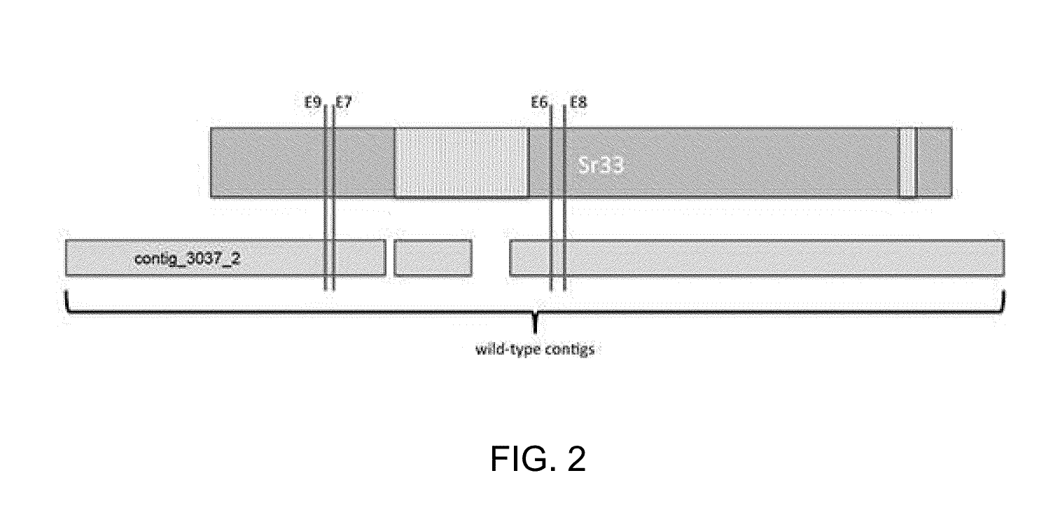

The longest contig had high homology to the bread wheat (Triticum aestivum) mitochondrial genome (data not shown). In order to restrict subsequent analysis to target regions, the bait sequences were aligned to the assembly. Every region in any contig that had an alignment to one of the bait sequences was considered for further analysis. An alignment of the known Sr33 genomic locus to the wildtype assembly revealed three contigs with 100% identity to the gene (FIG. 1). All exons of the Sr33 gene were completely represented except the last 33 bp of the first exon. More importantly, the positions of the four point mutants published previously were represented in the assembly (FIG. 2).

### Define NB-LRR Regions: In Silico Capture

Although the sequences captured by the baits are highly enriched, a de novo assembly will nevertheless assemble off-target sequence. In fact, less than 5% of our de novo assembled sequence had homology to known NB-LRR genes or source sequences. The rest is considered as flanking sequence or off-target sequence. It is likely that sequenced mutants do not have the same off-target sequences. Therefore this part would be identified as zero-coverage/deletion mutant. To avoid this, all bait sequences are aligned to the wild-type assembly. Only regions of the wild-type assembly with homology to a known NB-LRR or a bait source sequence were considered for further analysis.

### Mapping of Mutant Lines

Paired-end raw data of mutant lines were mapped to the wild-type assembly using BWA, version 0.7.4. Only reads mapping as a proper pair were used for further analysis. This selection as well as the subsequent pileup of reads was done using samtools version 0.1.19.

### Command Line Calls for Programs

bwa mem wildtype_Sr33.fasta E9_R1.fastq E9_R2.fastq|samtools view -Shub -f 2 -o E9.intermediate.bam -;
samtools sort E9.intermediate.bam E9;
samtools index E9.bam;
samtools mpileup -f wildtype_Sr33.fasta E9.bam >E9.pileup;

### Identification of Candidate Contigs

For each identified NB-LRR region the number of identified SNPs within each mutant line and the coverage of the region with paired reads from the mutant line are recorded. Different SNP calling methods may be applied. In case of Sr33, even the most basic method was sufficient to identify exactly one candidate contig. Here, a “SNP” was only defined by a reference allele frequency of maximal 10% and a minimum read coverage of 30 reads.

Coverage is recorded per base of the NB-LRR region. Several methods can be applied to identify a deletion mutant. The easiest way is to monitor the coverage at that base position where other mutant lines have a SNP. Another possibility is to apply a minimum coverage threshold for the entire region. The potential danger with this second method is the identification of a deletion mutant due to a false positive NB-LRR region.

It is most unlikely that different mutant lines have a point mutation at the very same position. This assumption can be utilized to minimize the number of false positive SNPs. At sufficient sequencing coverage (>50×), false positive SNPs are caused by mis-assembled contigs of the wild-type rather than sequencing errors of the mutant lines. Therefore, SNPs that are identified in more than one mutant line are discarded as false positives. This step in the pipeline can easily be switched off in case no candidate contig is identified. However, both methods were successful in identifying Sr33.

Finally, a candidate NB-LRR region is identified if each mutant line is either a deletion mutant for this region or has at least one SNP in this region. This approach was sufficient to identify the single contig representing the 5′ exon of Sr33 from the three sequenced mutants.

Potentially, the pipeline can end with more than one candidate. In that case, this number can be further minimized by identifying the correct open reading frame and filter out contigs with synonymous SNPs. This can easily be done by aligning candidate contigs to known NB-LRR protein sequences. Another problem might occur if mutations from different lines are in different exons separated into distinct contigs during the wild-type assembly. Three approaches can be applied to tackle this potential problem:
1. Improve the wild-type assembly using long-read technologies (e.g. Pacific Biosciences).
2. Sequence additional mutant lines.
3. Merge candidate contigs. Contigs can be identified as being potential parts of the same protein by alignment to a closely related NB-LRR protein sequence or by genetic mapping.
4. Perform RenSeq on the NB-LRR transcriptome of the parent and/or mutagenized plants.

### Simulation of Illumina HiSeq Sequencing of Mutant Lines

In our pilot study, mutant lines were sequenced using MiSeq and a read length of 250 bp PE reads. Although longer reads for the HiSeq2000 have been announced, the current read length of this particular platform is 150 bp PE reads. The MiSeq raw data was artificially clipped to a read length of 150 bp PE reads (to simulate HiSeq reads) and the pipeline described above was repeated. This again identified the single contig representing the 5′ end of Sr33. The SNPs of E9 and E7 as well as the zero coverage of the deletion mutant E5 were correctly identified.

### Example 2

### RenSeq is Compatible with Next-Generation Sequencing Technologies with Longer Reads

To improve de novo assembly of NB-LRR genes and detect polymorphisms linked to disease resistance, we refined our RenSeq method to be compatible with two technologies offering longer reads, the Illumina MiSeq system (Illumina, Inc., San Diego, Calif., USA), and the PacBio RS II system (Pacific Biosciences of California, Inc., Menlo Park, Calif., USA).

Sample preparation for MiSeq is as described for GAII with minor modifications. gDNA was sheared to 500-1000 bp fragments, followed by library preparation using NEBnext DNA library kit for Illumina, and enrichment. After hybridization and amplification, additional agarose gel-based size selection was applied to the library, to select fragments ranging from 600 to 900 bp. Libraries are then sequenced on a MiSeq Benchtop Sequencer with 250 bp paired end reads, with up to 12 single samples multiplexed. Application of the same workflow to cDNA and analysis of only expressed genes allowed a reduction of the number of candidate NB-LRRs by 50% in tomato, because ˜half of NB-LRR-encoding genes are not expressed. The combination of longer MiSeq reads and our published RenSeq pipeline (Jupe et al., 2013, Plant J. 76(3):530-44) reduced the background in SNP calling and improved de novo assembly compared to the previously used 76 bp sequencing libraries. Longer reads also led to the assembly of full-length NB-LRRs (˜5%), mostly NB-LRRs without paralogues.

For an improved de novo assembly and more precise assignment of polymorphisms to paralogues differing by only a few nucleotides, we adopted the RenSeq protocol for PacBio sequencing. Due to the high error rate of long PacBio reads (12-15%), we tested the self-correcting Circular Consensus Sequencing (CCS) of 1.5-2 kb fragments. DNA was sheared to ˜2 kb fragments, followed by an additional size selection with AMPure beads. Mixing the beads in a ratio of 0.45:1 to DNA allowed the selection of DNA fragments greater than 1.3 kb. The elimination of shorter fragments is vital in this protocol to enhance amplification of the larger target fragments. The libraries were also prepared using the NEBnext Illumina kit (Illumina, Inc., San Diego, Calif., USA) with barcoded Illumina adaptors (Illumina, Inc., San Diego, Calif., USA). Hybridization and amplification was carried out as for GAII/MiSeq libraries. The number of post-hybridization PCR cycles needs to be increased for a final yield of >1 ug of DNA. An additional agarose gel-based size selection was performed after the amplification, to select fragments of 1.4-2 kb. This DNA library was supplied to the PacBio sequencing service provider. One SMRT cell generated 13,000 CCS reads with length between 1.4 and 2 kb and an accuracy quality of >98%. Analysis showed that at least 50% of the reads are on target. The assembly of the PacBio data using PacBio software HGAP generates around 300 NB-LRR encoding contigs with length between 2 to 5 kb. Currently, we are using MIRA, Celera and CLC Assembly Cell to generate de novo assemblies with high coverage MiSeq 250 bp data (>200×) and with long low coverage PacBio reads (5-10×), although the present invention does not depend on the use of a particular method of assembly. Any method for generating de novo sequence assemblies that is described elsewhere herein or otherwise known in the art can be used in the methods of the present invention.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for identifying a plant resistance (R) gene for a plant disease of interest, the method comprising:
(a) selecting a subgroup of nucleic acids comprising
(i) hybridizing in solution a group of nucleic acids and a set of bait sequences to form a hybridization mixture, wherein the group of nucleic acids are derived from a mutagenized plant that is susceptible to the plant disease of interest and wherein the bait sequences are designed to hybridize to one or more genes from at least one R gene family, and
(ii) isolating from the hybridization mixture a subgroup of the nucleic acids that are hybridized to the bait sequences from nucleic acids that are not hybridized to the bait sequences;
(b) sequencing the subgroup of nucleic acids to obtain a collection of nucleic acid sequences;
(c) comparing the nucleic acid sequences obtained in (b) with corresponding sequences of the one or more genes that are derived from a reference plant that is resistant to the plant disease of interest; and
(d) identifying at least one nucleic acid sequence derived from the mutagenized plant that is not identical in sequence to a corresponding sequence from the reference plant, wherein the corresponding sequence comprises a nucleic acid sequence of at least a portion of an R gene for the plant disease of interest.

2. The method of claim 1, further comprising performing steps (a)-(d) one of more additional times, wherein each additional time the group of nucleic acids is derived from a different mutagenized plant.

3. The method of claim 1, wherein the mutagenized plant or plants and the reference plant are in the same genus or are the same species.

4. The method of claim 1, wherein the mutagenized plant or plants is/are produced by mutagenizing at least one plant of the same genotype as the reference plant.

5. The method of claim 1, wherein the mutagenized plant or plants was/were produced by mutagenesis comprising exposing at least one plant to a chemical mutagen or radiation.

6. The method of claim 5, wherein the chemical mutagen is selected from the group consisting of ethyl methanesulfonate (EMS), di-epoxy-butane (DEB), and sodium azide.

7. The method of claim 1, wherein the mutagenized plant or plants was/were produced by mutagenesis comprising exposing at least one plant that is resistant to the plant disease of interest to a mutagen and selecting at least one progeny plant that is susceptible to the plant disease of interest.

8. The method of claim 1, further comprising selecting at least one mutagenized plant from a population of mutagenized plants that was produced by exposing plants that are resistant to the plant disease of interest to an effective amount of a mutagen, wherein selecting comprises screening the population for susceptibility to the plant disease of interest and identifying at least one at least one mutagenized plant that is susceptible to the plant disease of interest.

9. The method of claim 1, further comprising producing at least one mutagenized plant by exposing plants that are resistant to the plant disease of interest to an effective amount of a mutagen to produce a population of mutagenized plants and selecting from the population at least one plant that is susceptible to the plant disease of interest.

10. The method of claim 7, wherein the plant that is resistant to the plant disease of interest is selected from the group consisting of, the reference plant, a plant comprising the same genotype as the reference plant, a plant comprising the same species as the reference plant, and a plant comprising the same species as the reference plant.

11. The method of claim 1, further comprising before step (c), obtaining the corresponding sequences by,
selecting a reference subgroup of nucleic acids comprising hybridizing in solution a reference group of nucleic acids and the set of bait sequences to form a reference hybridization mixture, wherein the reference group of nucleic acids are derived the reference plant,
isolating from the reference hybridization mixture a reference subgroup of nucleic acids that are hybridized to the bait sequences from the reference nucleic acids that are not hybridized to the bait sequences and/or from any non-hybridized bait sequences, and
sequencing the subgroup of reference nucleic acids to obtain a reference collection of nucleic acid sequences, wherein the reference collection of nucleic acid sequences comprises the one or more corresponding sequences.

12. The method of claim 1, wherein at least one of the mutagenized plant and the reference plant is a crop plant or a non-domesticated plant in the same species, genus, or family as at least one crop plant.

13. The method of claim 1, wherein the R gene encodes an NB-LRR protein.

14. The method of claim 1, wherein the sequencing is next-generation sequencing.

15. The method of claim 1, wherein the isolating step of (a)(ii) comprises contacting the hybridization mixture with at least one molecule or particle that binds to or is capable of separating the set of bait sequences from the hybridization mixture, and separating the set of bait sequences from the hybridization mixture to isolate a subgroup of nucleic acids that hybridize to the bait sequences from the group of nucleic acids.

16. The method of claim 11, wherein the isolating of the reference nucleic acids that are hybridized to the bait sequences comprises contacting the reference hybridization mixture with the molecule or particle that binds to or is capable of separating the set of bait sequences from the reference hybridization mixture, and separating the set of bait sequences from the reference hybridization mixture to isolate a subgroup of reference nucleic acids that hybridize to the bait sequences from the group of reference nucleic acids.

17. The method of claim 1, wherein the bait sequences are polynucleotides between about 60 nucleotides and 180 nucleotides in length.

18. The method of claim 1, wherein each of the bait sequences is designed to hybridize with a part of at least one member of the R gene family.

19. The method of claim 1, wherein each of the bait sequences is designed to be at least 80% identical to a part of the coding region of a member of the R gene family.

20. A method for identifying a plant resistance (R) gene for a plant disease of interest, the method comprising:
(a) producing at least one mutagenized plant by exposing plants that are resistant to the plant disease of interest to an effective amount of a mutagen to produce a population of mutagenized plants and selecting from the population at least one plant that is susceptible to the plant disease of interest;
(b) selecting a subgroup of nucleic acids comprising
(i) hybridizing in solution a group of nucleic acids and a set of bait sequences to form a hybridization mixture, wherein the group of nucleic acids are derived from the mutagenized plant and wherein the bait sequences are designed to hybridize to one or more genes from at least one R gene family, and
(ii) isolating from the hybridization mixture the subgroup of the nucleic acids that are hybridized to the bait sequences from nucleic acids that are not hybridized to the bait sequences, wherein the bait sequences are designed to hybridize to one or more genes from at least one R gene family;
(c) sequencing the subgroup of nucleic acids to obtain a collection of nucleic acid sequences;
(d) comparing the nucleic acid sequences obtained in (c) with corresponding sequences of the one or more genes that are derived from a reference plant that is resistant to the plant disease of interest; and
(e) identifying at least one nucleic acid sequence derived from the mutagenized plant that is not identical in sequence to a corresponding sequence from the reference plant, wherein the corresponding sequence comprises a nucleic acid sequence of at least a portion of an R gene for the plant disease of interest.

21. A method for identifying a gene associated with a phenotypic change for a trait of interest, the method comprising:
(a) selecting a subgroup of nucleic acids comprising
(i) hybridizing in solution a group of nucleic acids and a set of bait sequences to form a hybridization mixture, wherein the group of nucleic acids are derived from a mutagenized organism that comprises a phenotypic change for the trait of interest relative to the phenotype of the trait of interest for a reference organism, wherein the phenotypic change is induced by mutagenesis, and wherein the bait sequences are designed to hybridize to genes from a group or family of genes in the reference organism, and
(ii) isolating from the hybridization mixture a subgroup of nucleic acids that are hybridized to the bait sequences from any nucleic acids that are not hybridized to the bait sequences;
(b) sequencing the subgroup of nucleic acids to obtain a collection of nucleic acid sequences;
(c) comparing the nucleic acid sequences obtained in (b) with corresponding sequences of the group or family of genes that are derived from the reference organism; and
(d) identifying at least one nucleic acid sequence derived from the mutagenized organism that is not identical in sequence to a corresponding sequence from the reference organism, wherein the non-identical sequence comprises at least a portion of a nucleic acid sequence of a gene associated with a phenotypic change of interest.
